(19) United States
(12) Patent Application Publication
(10) Pub. No.: US 2023/0221325 A1
(43) Pub. Date: Jul. 13, 2023

(54) SOLUBLE CD28 LEVELS AFTER IMMUNOTHERAPY

(71) Applicant: BIOND BIOLOGICS LTD., Misgav, IL (US)

(72) Inventors: Anna FRIDMAN-DROR, Kibbutz Dalia (IL); Tal GABAY, Atlit (IL); Orit SHILOVIZKY, Haifa (IL); Dror ALISHEKEVITZ, Kiryat Tivon (IL); Ilana MANDEL, Manof (IL); Tehila BEN-MOSHE, Tel Aviv (IL); Yair SAPIR, Manof (IL); Avidor SHULMAN, Rakefet (IL); Motti HAKIM, Kibbutz Gazit (IL)

(21) Appl. No.: 18/124,717

(22) Filed: Mar. 22, 2023

Related U.S. Application Data
(63) Continuation-in-part of application No. 17/781,966, filed on Jun. 2, 2022.
(60) Provisional application No. 63/322,449, filed on Mar. 22, 2022.
(60) Provisional application No. 62/942,276, filed on Dec. 2, 2019.

(51) Int. Cl. G01N 33/574; A61P 35/00; C07K 16/28

(52) U.S. Cl. CPC G01N 33/57488; A61P 35/00; C07K 16/2818; A61K 2039/545

(57) ABSTRACT

Methods of determining suitability of a subject suffering from cancer or at risk of cancer relapse to receive treatment comprising an agent that reduces sCD28 levels are provided. Methods of treating a subject suffering from cancer or at risk of cancer relapse comprising administering an anti-PD-1/PD-L1 immunotherapy, measuring soluble CD28 levels in a subject, and administering an agent that reduces sCD28 levels to a subject whose sCD28 levels increased are also provided.

### CROSS REFERENCE TO RELATED APPLICATIONS

This application is a Continuation-In-Part of U.S. patent application Ser. No. 17/781,966, filed Jun. 2, 2022, which is a National Phase of PCT Patent Application PCT/IL2020/051244, filed Dec. 2, 2020, which claims the benefit of priority of U.S. Provisional Patent Application No. 62/942,276, filed Dec. 2, 2019.

This application also claims the benefit of priority of U.S. Provisional Patent Application No. 63/322,449, filed Mar. 22, 2022, the contents of which are all incorporated herein by reference in their entirety.

### REFERENCE TO AN ELECTRONIC SEQUENCE LISTING

The contents of the electronic sequence listing (BDB-P-010-US1.xml; Size: 107,922 bytes; and Date of Creation: Mar. 19, 2023) is herein incorporated by reference in its entirety.

### FIELD OF INVENTION

The present invention is in the field of immune regulation and immunotherapy.

### BACKGROUND OF THE INVENTION

It is known that some co-stimulatory molecules have several physiological forms. Alongside membrane-bound forms, soluble forms have been described that are expressed in naive immune cells, increasing the complexity of T cell biology. The soluble form of CD28 (sCD28) has been implicated in inhibiting an immune response to cancer. Further, sCD28 has been demonstrated to suppress the effectiveness of PD-1 and PD-L1 based immunotherapies. It has been hypothesized that sCD28's presence may be a contributing factor to the heterogeneity of response to immunotherapy.

However, the status of sCD28 during the course of immunotherapy and its targeting to enhance immunotherapy has not been investigated. Further, the efficacy of sCD28 removal/blockade in patients that are refractory to immunotherapeutic treatment is unknown.

### SUMMARY OF THE INVENTION

The present invention provides methods of treating cancer in a subject and of determining suitability of subject suffering from cancer to receive treatment. The methods comprise measuring soluble CD28 (sCD28) levels in the subject, wherein an increase in sCD28 levels indicates the subject is suitable for treatment with an agent that reduces sCD28 levels and treating such a subject.

According to a first aspect, there is provided a method of determining suitability of a subject suffering from cancer or at risk of cancer relapse to receive a treatment comprising an agent that reduces sCD28 levels, the method comprising receiving measurements of sCD28 levels in the subject at at least two time points, wherein at least a first time point is before administering an anti-PD-1/PD-L1 immunotherapy to the subject and at least a second time point is after the administering, wherein an increase in sCD28 levels from before the administering to after by more than a predetermined threshold indicates the subject is suitable to receive the treatment, thereby determining suitability.

According to some embodiments, the method further comprises administering the anti-PD-1/PD-L1 immunotherapy between the first and second time points.

According to another aspect, there is provided a method of treating a subject suffering from cancer or at risk of cancer relapse, the method comprising administering an anti-PD-1/PD-L1 immunotherapy to the subject, measuring soluble CD28 (sCD28) levels in the subject at at least two time points wherein at least a first time point is before the administering and at least a second time point is after the administering, and further administering an agent that reduces sCD28 levels to a subject whose sCD28 levels increased from before administering to after administering by at least a predetermined threshold, thereby treating a subject suffering from cancer.

According to some embodiments, the second time point is at least 7 days after the administering.

According to some embodiments, an increase in sCD28 levels by more than the predetermined threshold indicates the subject is not responding or unlikely to respond to the anti-PD-1/PD-L1 immunotherapy.

T According to some embodiments, the administering an agent that reduces sCD28 levels converts a not responding subject to a responding subject.

According to some embodiments, the predetermined threshold is 1 ng/ml sCD28, a 10% increase or both.

According to some embodiments, the method further comprises increasing a dose of the PD-1/PD-L1 based immunotherapy administered to the subject.

According to some embodiments, the treatment comprising an agent that reduces sCD28 levels comprises performing a method of the invention.

According to some embodiments, the increase is an increase of at least 25%.

According to some embodiments, the increase is an increase to at least 1 ng/mL sCD28.

According to some embodiments, the measuring comprises obtaining a sample from the subject and measuring sCD28 levels in the sample.

According to some embodiments, the sample is a blood sample.

According to some embodiments, the cancer is selected from skin cancer, urothelial cancer, lung cancer, colon cancer and renal cancer.

According to some embodiments, the agent that reduces sCD28 levels is an agent that binds sCD28 and degrades the sCD28 or targets the sCD28 for degradation.

According to some embodiments, the agent is an antibody or antigen binding fragment thereof and comprises three heavy chain CDRs (CDR-H) and three light chain CDRs (CDR-L), wherein:
CDR-H1 comprises the amino acid sequence set forth in SEQ ID NO: 12 (GYTLTNY), CDR-H2 comprises the amino acid sequence as set forth in SEQ ID NO: 13 (NTYTGK), CDR-H3 comprises the amino acid sequence as set forth in SEQ ID NO: 14 (GDANQQFAY), CDR-L1 comprises the amino acid sequence as set forth in SEQ ID NO: 15 (KASQDINSYLS), CDR-L2 comprises the amino acid sequence as set forth in SEQ ID NO: 16 (RANRLVD), and CDR-L3 comprises the amino acid sequence as set forth in SEQ ID NO: 17 (LQYDEFPPT);
CDR-H1 comprises the amino acid sequence set forth in SEQ ID NO: 18 (GYTFTSY), CDR-H2 comprises the amino acid sequence as set forth in SEQ ID NO: 19 (YPGDGD), CDR-H3 comprises the amino acid sequence as set forth in SEQ ID NO: 20 (NYRYSSFGY), CDR-L1 comprises the amino acid sequence as set forth in SEQ ID NO: 21 (KSSQSLLNSGNQKNYLT), CDR-L2 comprises the amino acid sequence as set forth in SEQ ID NO: 22 (WASTRES), and CDR-L3 comprises the amino acid sequence as set forth in SEQ ID NO: 23 (QSDYSYPLT); or
CDR-H1 comprises the amino acid sequence set forth in SEQ ID NO: 24 (GYTFTDY), CDR-H2 comprises the amino acid sequence as set forth in SEQ ID NO: 25 (NPNYDS), CDR-H3 comprises the amino acid sequence as set forth in SEQ ID NO: 26 (SSPYYDSNHFDY), CDR-L1 comprises the amino acid sequence as set forth in SEQ ID NO: 27 (SARSSINYMH), CDR-L2 comprises the amino acid sequence as set forth in SEQ ID NO: 28 (DTSKLAS), and CDR-L3 comprises the amino acid sequence as set forth in SEQ ID NO: 29 (HQRNSYPFT).

According to some embodiments, the agent that reduces sCD28 levels is an agent that binds membranal CD28 (mCD28) and inhibits proteolytic cleavage of the mCD28.

According to some embodiments, the agent does not degrade mCD28 or decrease mCD28-mediated immune cell activation.

According to some embodiments, the agent binds mCD28 in a stalk region and occludes an MMP-2, MMP-13 or both cleavage site, PX1X2/X3 wherein X3 is a hydrophobic residue, in the stalk region.

According to some embodiments, the cleavage site is PSPL and the MMP-2, MMP-13 or both cleaves between the P and the L.

According to some embodiments, the agent is an antibody or antigen binding fragment thereof and comprises three heavy chain CDRs (CDR-H) and three light chain CDRs (CDR-L), wherein:
CDR-H1 comprises the amino acid sequence set forth in SEQ ID NO: 30 (GFTFSSYYMS), CDR-H2 comprises the amino acid sequence as set forth in SEQ ID NO: 31 (TISDGGDNTYYAGTVTG), CDR-H3 comprises the amino acid sequence as set forth in SEQ ID NO: 32 (IHWPYYFDS), CDR-L1 comprises the amino acid sequence as set forth in SEQ ID NO: 33 (RASSSVSYMN), CDR-L2 comprises the amino acid sequence as set forth in SEQ ID NO: 34 (ATSDLAS), and CDR-L3 comprises the amino acid sequence as set forth in SEQ ID NO: 35 (QQWSSHPPT).

According to some embodiments, the agent is a single domain antibody.

According to some embodiments, the single domain antibody comprises three CDRs wherein:
CDR1 comprises the amino acid sequence set forth in SEQ ID NO: 57 (INAMG),
CDR2 comprises the amino acid sequence as set forth in SEQ ID NO: 58 (AISGGGDTYYADSVKG), CDR3 comprises the amino acid sequence as set forth in SEQ ID NO: 59 (DLYGSDYWD);
CDR1 comprises the amino acid sequence set forth in SEQ ID NO: 36 (INAMA),
CDR2 comprises the amino acid sequence as set forth in SEQ ID NO: 37 (AITSSGSTNYANSVKG), CDR3 comprises the amino acid sequence as set forth in SEQ ID NO: 38 (DEYGSDYWI);
CDR1 comprises the amino acid sequence set forth in SEQ ID NO: 57 (INAMG),
CDR2 comprises the amino acid sequence as set forth in SEQ ID NO: 39 (AITSGGSTNYADSVKG), CDR3 comprises the amino acid sequence as set forth in SEQ ID NO: 40 (DLYGEDYWI);
CDR1 comprises the amino acid sequence set forth in SEQ ID NO: 74 (INSMG),
CDR2 comprises the amino acid sequence as set forth in SEQ ID NO: 75 (AINEKLLIYYADSVKG), CDR3 comprises the amino acid sequence as set forth in SEQ ID NO: 59 (DLYGSDYWD);
CDR1 comprises the amino acid sequence set forth in SEQ ID NO: 57 (INAMG),
CDR2 comprises the amino acid sequence as set forth in SEQ ID NO: 58 (AISGGGDTYYADSVKG), CDR3 comprises the amino acid sequence as set forth in SEQ ID NO: 76 (DMIEQQWWY);
CDR1 comprises the amino acid sequence set forth in SEQ ID NO: 57 (INAMG),
CDR2 comprises the amino acid sequence as set forth in SEQ ID NO: 58 (AISGGGDTYYADSVKG), CDR3 comprises the amino acid sequence as set forth in SEQ ID NO: 77 (DTHRGVYWY);
CDR1 comprises the amino acid sequence set forth in SEQ ID NO: 78 (IKTMA),
CDR2 comprises the amino acid sequence as set forth in SEQ ID NO: 79 (AINYIKEIYYADSVKG), CDR3 comprises the amino acid sequence as set forth in SEQ ID NO: 80 (DVTKEDYWY);
CDR1 comprises the amino acid sequence set forth in SEQ ID NO: 81 (INSMA),
CDR2 comprises the amino acid sequence as set forth in SEQ ID NO: 82 (AISNAREVYYADSVKG), CDR3 comprises the amino acid sequence as set forth in SEQ ID NO: 83 (DVYFQEYWY);
CDR1 comprises the amino acid sequence set forth in SEQ ID NO: 84 (INTMA),
CDR2 comprises the amino acid sequence as set forth in SEQ ID NO: 85 (AINSISRTYYADSVKG), CDR3 comprises the amino acid sequence as set forth in SEQ ID NO: 80 (DVTKEDYWY);
CDR1 comprises the amino acid sequence set forth in SEQ ID NO: 78 (IKTMA),
CDR2 comprises the amino acid sequence as set forth in SEQ ID NO: 86 (AIASDNRKYYADSVKG), CDR3 comprises the amino acid sequence as set forth in SEQ ID NO: 80 (DVTKEDYWY);
CDR1 comprises the amino acid sequence set forth in SEQ ID NO: 87 (IRTMA),
CDR2 comprises the amino acid sequence as set forth in SEQ ID NO: 88 (AISSGREVYYADSVKG), CDR3 comprises the amino acid sequence as set forth in SEQ ID NO: 89 (DMYWQDYWW); or
CDR1 comprises the amino acid sequence set forth in SEQ ID NO: 74 (INSMG),
CDR2 comprises the amino acid sequence as set forth in SEQ ID NO: 90 (AISDRSEKYYADSVKG), CDR3 comprises the amino acid sequence as set forth in SEQ ID NO: 91 (DHHHSDWWT).

According to some embodiments, the agent further comprises a moiety that increases stability of the agent.

According to some embodiments, the agent that reduces sCD28 levels is a matrix metalloprotease (MMP) inhibitor.

According to some embodiments, the MMP is MMP-2, MMP-13 or both.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Survival chart of melanoma patients undergoing anti-PD1 therapy. Patients with a low level of sCD28 before initiation of the therapy had a longer mean survival than patients with high levels of sCD28.

FIGS. 2A-2D: (2A) Line graphs of the levels of sCD28 in 17 patients (9 responders and 8 non-responders) over the course of several weeks of immunotherapy. (2B-2C) Bar graphs of mean sCD28 levels in (2B) non-responders and (2C) responders to immunotherapy at three time points. (2D) Bar graph of absolute changes in sCD28 expression from the initial reading before initiation of immunotherapy to two time points after initiation. *is a Pval=0.06, **is a Pval<0.05 according to the Signed Rank test.

FIGS. 3A-3B: (3A) Line graphs of sCD28 levels from two patients that responded to immunotherapy and whose cancer then relapsed. CR is complete response; PR is partial response; PD is progressive disease; and SD is stable disease. (3B) Scatter plot of the greatest change in sCD28 levels as compared to the initial reading before initiation of immunotherapy for each subject during the course of measurements (6 months). N=66; ***is a Pval=0.0001 according to the Mann-Whitney test.

FIGS. 4A-4C: Anti-PD-1 treatment increases soluble CD28 accumulation by proteolytic CD28 shedding. (4A) Line graphs of sCD28 levels in culture media of SEB stimulated human PBMC (from two different healthy donors) treated with either isotype control (IC, light grey line, circles) or anti-PD-1 (Keytruda, black line, squares) that were sampled over time. Naïve unstimulated cells were also sampled (dark grey line, triangles). (4B) Bar graph of sCD28 in culture media of 7 days CMV-stimulated PBMC (light grey bar), CMV stimulation in the presence of anti-PD-1 (Keytruda, black bar) and CMV stimulation in the presence of both anti-PD-1 and a MMP specific inhibitor (GI254023X, dark grey bar). (4C) Bar graph of sCD28 quantification in 6-day allogenic mixed lymphocytes reaction of mature DC and CD3 T cells (light grey bar), MLR in the presence of anti-PD-1 (Keytruda, black bar) and MLR in the presence of both anti-PD-1 and anti-CD28 VHH-hFc (dark grey bar). The concentration of human sCD28 in the supernatants was quantified with standardized sandwich ELISA (R&D system). *unpaired T.Test at P<0.05 compared to isotype control only.

FIGS. 5A-5D: Reduced anti-PD-1 efficacy is observed in CD28-shedding permissive mice. (5A) Bar graph of serum levels of sCD28 quantified weekly during the study illustrating the effect of anti-PD-1 on soluble CD28 generation. *Log-rank test, WT vs hCD28Tg anti-PD-1 arms. **T.Test at P<0.05 compared to isotype control treatment. (5B-5C) Line graphs of (5B) tumor volume and (5C) survival rate of MC-38 colon cancer cells grown in C57b WT (black lines) and hCD28Tg (grey lines) mice treated with either isotype control (straight lines) or anti-mouse PD-1 antibody clone RMP1-14 (dashed lines). (5D) Tumor growth spiders-plots of anti-PD-1 treated WT and hCD28 transgenic mice show higher relapse cases in CD28-shedding permissive mice (left and middle plot). Relapse cases were plotted as part of response incidence in both mice (right chart).

### DETAILED DESCRIPTION OF THE INVENTION

The present invention, in some embodiments, provides methods of treating a subject suffering from cancer by administering an anti-PD-1/PD-L1 immunotherapy, comprising measuring soluble CD28 (sCD28) before and after the administering and further administering an agent that reduces sCD28 levels to a subject whose sCD28 levels increased from before to after by at least a predetermined threshold. The invention further provides methods of determining suitability of a subject suffering from cancer to receive a treatment with an agent that reduces sCD28 levels, comprising measuring soluble sCD28 before and after the administering wherein an increase in sCD28 levels from before to after by at least a predetermined threshold indicates the subject is suitable to receive the treatment. The invention is based on the surprising finding that immune cells in subjects that receive anti-PD-1/PD-L1 immunotherapy can produce increased levels of sCD28 which inhibit function of the immunotherapy, but that this increase can be abrogated by an agent that reduces sCD28. It is further based on the surprising finding that this sCD28 levels increase can confer resistance onto a subject which was initially responding and thus cause relapse of the cancer, which also can be abrogated by an agent that reduces sCD28 levels.

By a first aspect, there is provided a method of treating a subject, the method comprising administering an immunotherapy to the subject, measuring soluble CD28 (sCD28) levels in the subject at at least two time points wherein at least a first time point is before the administering and at least a second time point is after the administering and further administering an agent that reduces sCD28 levels to a subject whose sCD28 levels increased by at least a predetermined threshold from the first time point to the second time point; thereby treating a subject.

By another aspect, there is provided a method of determining suitability of a subject to receive a treatment comprising an agent that reduces sCD28 levels, the method comprising receiving measurements of sCD28 levels in the subject at at least 2 time points, wherein at least a first time point is before administering an anti-PD-1/PD-L1 immunotherapy to the subject and at least a second time point is after the administering, wherein an increase in sCD28 levels by at least a predetermined threshold from the first time point to the second time point indicates the subject is suitable to receive the treatment; thereby determining suitability .

By another aspect, there is provided a method of preventing cancer relapse, the method comprising administering to a subject in need thereof an agent that reduces sCD28 levels, thereby preventing cancer relapse.

In some embodiments, the CD28 is mammalian CD28. In some embodiments the CD28 is human CD28. In some embodiments, the human CD28 comprises or consists of the amino acid sequence:

(SEQ ID NO: 1)
MLRLLLALNLFPSIQVTGNKILVKQSPMLVAYDNA
VNLSCKYSYNLFSREFRASLHKGLDSAVEVCVVYG
NYSQQLQVYSKTGFNCDGKLGNESVTFYLQNLYVN
QTDIYFCKIEVMYPPPYLDNEKSNGTIIHVKGKHL
CPSPLFPGPSKPFWVLVVVGGVLACYSLLVTVAFI
IFWVRSKRSRLLHSDYMNMTPRRPGPTRKHYQPYA
PPRDFAAYRS.

In some embodiments, mature CD28 lacks a signal peptide and comprises the sequence:

(SEQ ID NO: 2)
NKILVKQSPMLVAYDNAVNLSCKYSYNLFSREFRA
SLHKGLDSAVEVCVVYGNYSQQLQVYSKTGFNCDG
KLGNESVTFYLQNLYVNQTDIYFCKIEVMYPPPYL
DNEKSNGTIIHVKGKHLCPSPLFPGPSKPFWVLVV
VGGVLACYSLLVTVAFIIFWVRSKRSRLLHSDYMN
MTPRRPGPTRKHYQPYAPPRDFAAYRS.

In some embodiments, the DNA coding sequence that codes for full length human CD28 comprises the sequence:

(SEQ ID NO: 3)
ATGCTCAGGCTGCTCTTGGCTCTCAACTTATTCCC
TTCAATTCAAGTAACAGGAAACAAGATTTTGGTGA
AGCAGTCGCCCATGCTTGTAGCGTACGACAATGCG
GTCAACCTTAGCTGCAAGTATTCCTACAATCTCTT
CTCAAGGGAGTTCCGGGCATCCCTTCACAAAGGAC
TGGATAGTGCTGTGGAAGTCTGTGTTGTATATGGG
AATTACTCCCAGCAGCTTCAGGTTTACTCAAAAAC
GGGGTTCAACTGTGATGGGAAATTGGGCAATGAAT
CAGTGACATTCTACCTCCAGAATTTGTATGTTAAC
CAAACAGATATTTACTTCTGCAAAATTGAAGTTAT
GTATCCTCCTCCTTACCTAGACAATGAGAAGAGCA
ATGGAACCATTATCCATGTGAAAGGGAAACACCTT
TGTCCAAGTCCCCTATTTCCCGGACCTTCTAAGCC
CTTTTGGGTGCTGGTGGTGGTTGGTGGAGTCCTGG
CTTGCTATAGCTTGCTAGTAACAGTGGCCTTTATT
ATTTTCTGGGTGAGGAGTAAGAGGAGCAGGCTCCT
GCACAGTGACTACATGAACATGACTCCCCGCCGCC
CCGGGCCCACCCGCAAGCATTACCAGCCCTATGCC
CCACCACGCGACTTCGCAGCCTATCGCTCCTGA.

As used herein, sCD28 refers to any CD28 fragment or variant that does not comprise a transmembrane domain and thus cannot be integrated in a membrane. In some embodiments, the CD28 transmembrane domain comprises the amino acid sequence FWVLVVVGGVLACYSLLVTVAFIIFWV (SEQ ID NO: 4). In some embodiments, sCD28 is not membrane bound. In some embodiments, sCD28 is in solution. In some embodiments, the sCD28 is CD28 in blood. In some embodiments, the sCD28 is CD28 in the TME. In some embodiments, sCD28 is CD28 in a bodily fluid. In some embodiments, sCD28 is a cleavage product from membranal CD28 (mCD28). In some embodiments, sCD28 is truncated CD28. In some embodiments, sCD28 lacks the cytoplasmic domain of full-length CD28. In some embodiments, sCD28 is dimeric sCD28. In some embodiments, sCD28 is monomeric sCD28. In some embodiments, sCD28 is not a splice variant arising from alternative splicing of CD28. In some embodiments, sCD28 comprises the amino acid sequence: MLRLLLALNLFP SIQVTGNKILVKQ SPMLVAYDNAVNL SCKYSYNLF SREFRASLHKG LD SAVEVCVVYGNYSQQLQVYSKTGFNCDGKLGNESVTFYLQNLYVNQTDIYFCKIE VMYPPPYLDNEKSNGTIIHVKGKHLCPSP (SEQ ID NO: 5). In some embodiments, sCD28 lacks the signal peptide and comprises the sequence:

(SEQ ID NO: 6)
NKILVKQSPMLVAYDNAVNLSCKYSYNLFSREFRA
SLHKGLDSAVEVCVVYGNYSQQLQVYSKTGFNCDG
KLGNESVTFYLQNLYVNQTDIYFCKIEVMYPPPYL
DNEKSNGTIIHVKGKHLCPSP.

In some embodiments, the subject suffers from cancer. In some embodiments, the subject is naive to therapy. In some embodiments, the subject had cancer and is currently cancer free. In some embodiments, the subject has cancer that is in remission. In some embodiments, the subject has cancer that is in partial remission. In some embodiments, remission comprises complete remission and partial remission. In some embodiment remission comprises a partial response and a complete response. In some embodiments, remission comprises complete remission. In some embodiments, remission comprises partial remission. In some embodiments, the subject is at risk of cancer relapse. In some embodiments, the subject has a family history of relapse. In some embodiments, the subject is in need of relapse prevention. In some embodiments, the subject in need of relapse prevention was treated with an immunotherapy. In some embodiments, the immunotherapy produced cancer regression and the subject is at risk of relapse. In some embodiments, the subject has discontinued the immunotherapy. In some embodiments, the immunotherapy was discontinued due to cancer cure or cancer becoming undetectable. In some embodiments, the subject is still receiving the immunotherapy. In some embodiments, the subject has undergone therapy for the cancer. In some embodiments, the subject is undergoing therapy for the cancer. In some embodiments, the therapy is immunotherapy. In some embodiments, the subject is a responder to the therapy. In some embodiments, the subject is a non-responder to the therapy. In some embodiments, the immunotherapy is PD-1 based immunotherapy. In some embodiments, the immunotherapy is PD-L1/L2 based immunotherapy. In some embodiments, the immunotherapy is PD-L1 based immunotherapy. In some embodiments, the immunotherapy is PD-1/PD-L1 based immunotherapy. In some embodiments, the subject has undergone PD-1/PD-L1 based immunotherapy. In some embodiments, the subject is undergoing PD-1/PD-L1 based immunotherapy. In some embodiments, immunotherapy is blockade. In some embodiments, immunotherapy is immune checkpoint inhibition.

In some embodiments, a responder is a subject that is responsive to the therapy. In some embodiments, a responder is someone that is responding. In some embodiments, someone that is responding is a responder. In some embodiments, a responder is a subject with a favorable response to the therapy. As used herein, a “favorable response” of the cancer patient indicates “responsiveness” of the cancer patient to the treatment with the therapy, namely, the treatment of the responsive cancer patient with the immunotherapy will lead to the desired clinical outcome such as tumor regression, tumor shrinkage or tumor necrosis; an anti-tumor response by the immune system; preventing or delaying tumor recurrence, tumor growth or tumor metastasis. In some embodiments, response is complete response (CR). In some embodiments, response is partial response (PR). In some embodiments, the response is cancer regression. In some embodiments, regression is from stable disease. In some embodiments, regression is from progressive disease. In some embodiments, a non-responder is a subject that is not responsive to the therapy. In some embodiments, a non-responder is someone that is not responding. In some embodiments, a non-responder is someone that is unlikely to respond. In some embodiments, someone that is not responding is a non-responder. In some embodiments, a non-responder is a subject with a non-favorable response to the therapy. As used herein a “non-favorable response” of the cancer patient indicates “non-responsiveness” of the cancer patient to the treatment with the therapy and thus the treatment of the non-responsive cancer patient with the therapy will not lead to the desired clinical outcome, and potentially to a non-desired outcome such as tumor expansion, recurrence and metastases. In some embodiments, the non-desired outcome is cancer relapse. In some embodiments, a non-desired response is development of stable disease. In some embodiments, a non-desired response is development of progressive disease.

As used herein, the terms “complete response”, “partial response”, “stable disease” and “progressive disease” are all evaluation criteria for assessing cancer and in particular solid lesions. During evaluation, lesions are measured in order to provide basis for comparison and evaluation during treatment. In some embodiments, complete response refers to all lesions having disappeared during the course of treatment. In some embodiments, complete response refers to all target lesions having disappeared during the course of treatment. In some embodiments, partial response refers to a decrease in the size of a lesion or lesions. In some embodiments, the decrease is size is of the largest lesion. In some embodiments, the decrease is in a sum of the sizes of all the lesions. In some embodiments, the decrease is in the average size of the lesions. In some embodiments, the decrease in size is a decrease of at least 10, 20, 30, 40, or 50%. Each possibility represents a separate embodiment of the invention. In some embodiments, the decrease is size is a decrease of at least 30%. In some embodiments, the decrease is a decrease in the diameter of the lesion. In some embodiments, stable disease refers to no significant decrease or increase in the size of a lesion or lesions. In some embodiments, progressive disease refers to an increase in the size of the lesion or lesions. In some embodiments, the increase is in the largest lesion. In some embodiments, the increase is in a sum of the sizes of all the lesions. In some embodiments, the increase is in the average size of the lesions. In some embodiments, the increase in size is an increase of at least 10, 20, 30, 40, or 50%. Each possibility represents a separate embodiment of the invention. In some embodiments, the increase is size is an increase of at least 20%.

In some embodiments, the immunotherapy is PD-1 and/or PD-L1 based immunotherapy. In some embodiments, the immunotherapy is PD-1 based immunotherapy. In some embodiments, the immunotherapy is PD-L1 based immunotherapy. In some embodiments, the PD-1/PD-L1 based immunotherapy comprises administering an anti-PD1 or anti-PD-L1 antibody. In some embodiments, the therapy comprises blockade of the PD-1 checkpoint. In some embodiments, the immunotherapy comprises administering allogenic, syngenic or autologous immune cells to the subject. In some embodiments, the immune cells are T cells. In some embodiments, the subject in need of immunotherapy suffers from cancer. In some embodiments, PD-1 based immunotherapy comprises PD-L2 based immunotherapy. In some embodiments, PD-L1 based immunotherapy comprises PD-L2 based immunotherapy.

In some embodiments, the immunotherapy is CD80 based immunotherapy. In some embodiments, the immunotherapy is CD86 based immunotherapy. CD80 and CD86 immunotherapies are well known in the art and comprise administering CD80/CD86 and or mimic, derivatives or mimetics thereof to stimulate an immune response. CD8O-Fc is currently in clinical trials as an anticancer immunotherapeutic for non-limiting example.

In some embodiments, the immunotherapy is CTLA-4 based immunotherapy. In some embodiments, CTLA-4 immunotherapy comprises CTLA-4 blockade. In some embodiments, CTLA-4 immunotherapy comprises administering a CTLA-4 inhibitor. In some embodiments, CTLA-4 immunotherapy comprises administering an anti-CTLA-4 antibody. In some embodiments, the therapy comprises blockade of the CTLA-4 checkpoint. In some embodiments, the immunotherapy is a combination therapy of any of the herein described immunotherapies.

In some embodiments, the subject suffers from cancer. In some embodiments, the cancer is a cancer that can be treated with immunotherapy. In some embodiments, the cancer is a PD-L1 positive cancer. In some embodiments, the cancer is a PD-L2 positive cancer. In some embodiments, the cancer is a cancer that can be treated with PD-1/PD-L1 therapy. In some embodiments, the subject has undergone PD-1/PD-L1 therapy. In some embodiments, the subject is a non-responder to PD-1/PD-L1 therapy. In some embodiments, the subject is naive to PD-1/PD-L1 therapy. In some embodiments, the methods of the invention are performed together with PD-1/PD-L1 therapy. In some embodiments, the methods of the invention are performed before PD-1/PD-L1 therapy.

In some embodiments, the cancer is a cancer with elevated sCD28 levels. In some embodiments, the cancer comprises high sCD28 levels. In some embodiments, elevated and/or high sCD28 levels are levels at and/or above 5, 6, 7, 8, 9, 10, 12, 14, 15, 17, 20, 25, 30, 35, 40, 50, 60, 70, 80, 90 or 100 ng/mL. Each possibility represents a separate embodiment of the invention. In some embodiments, the cancer comprises high sCD28 levels. In some embodiments, elevated and/or high sCD28 levels are levels at and/or above 5, 10, 15, 20, 25, 30, 35, 40, 45, 50, 55, 60, 65, 70, 75, 80, 85, 90, 95, 100, 150, 200, 300, 400, 500, 600, 700, 800, 900, or 1000% of the levels in a healthy subject. Each possibility represents a separate embodiment of the invention. In some embodiments, the cancer is not breast cancer. In some embodiments, the cancer is selected from melanoma, urothelial carcinoma, head and neck, non-small cell lung cancer, ovarian, kidney, gastric and colorectal. In some embodiments, the cancer is selected from melanoma, urothelial carcinoma, head and neck, non-small cell lung cancer, ovarian, and colorectal. In some embodiments, the cancer is melanoma, urothelial carcinoma, head and neck, non-small cell lung cancer, ovarian, kidney, gastric or colorectal. Each possibility represents a separate embodiment of the invention. In some embodiments, the cancer is selected from melanoma and urothelial carcinoma. In some embodiments, the cancer is selected from skin cancer, urothelial cancer, lung cancer, and renal cancer. In some embodiments, the cancer is selected from skin cancer and urothelial cancer. In some embodiments, the cancer is colorectal cancer. In some embodiments, the cancer is colon cancer.

In some embodiments, sCD28 is measured at at least 1, 2, 3, 4, 5, 6, 7, 8, 9 or 10 time points. Each possibility represents a separate embodiment of the invention. In some embodiments, sCD28 is measured at at least 2 time points. In some embodiments, the at least two time points comprise a first time point and a second time point. In some embodiments, there is an increase from one time point to a second time point. In some embodiments, there is a decrease from one time point to a second time point. In some embodiments, there is no change from one time point to a second time point. In some embodiments, there is a trend in the sCD28 kinetics. In some embodiments, the trend is an increase or a decrease. In some embodiments, an increasing trend indicates the subject is not responsive to therapy. In some embodiments, an increasing trend indicates the subject is not likely to respond to therapy. In some embodiments, an increasing trend indicates suitability to receive treatment. In some embodiments, an increase from a first time point to a second indicates suitability to receive treatment. In some embodiments, treatment is with an agent that reduces sCD28 levels. In some embodiments, levels are levels in the subject. In some embodiments, in the subject is in the blood. In some embodiments, in the subject is in the tumor. In some embodiments, in the subject is in the tumor microenvironment (TME). In some embodiments, an agent that reduces sCD28 levels is administered to a subject with an increasing trend. In some embodiments, an agent that reduces sCD28 levels is administered to a subject with an increase from the first time point to the second. In some embodiments, from the first to the second time point is from before administering to after administering.

In some embodiments, a subject that responds to therapy is a responder. In some embodiments, a subject that does not respond to therapy is a non-responder. Response to therapy need not be a complete response but may be a partial response or a response of an improvement of at least one symptom. In some embodiments, a non-responder has no positive response to therapy.

In some embodiments, the increase is as compared to sCD28 levels in a healthy subject. In some embodiments, the increase is as compared to sCD28 levels in the subject before administering. In some embodiments, the increase is as compared to sCD28 levels in the subject before initiation of the immunotherapy. In some embodiments, the increase is as compared to a predetermined threshold. In some embodiments, the increase is as compared to levels before initiation of immunotherapy. In some embodiments, the increase is by at least a predetermined threshold. In some embodiments, the increase is a significant increase.

In some embodiments, the increase is from one time point to another. In some embodiments, the increase is from a first time point to a second time point. In some embodiments, an increase indicates the subject is a non-responder to therapy. In some embodiments, an increase indicates the subject is not responding to therapy. In some embodiments, an increase indicates the subject is suitable to receive an agent that reduces sCD28 levels. In some embodiments, an increase indicates the subject is administered an agent that reduces sCD28 levels. In some embodiments, the method further comprises increasing a dose to a not responding patient. In some embodiments, the increase is a statistically significant increase. In some embodiments, the method further comprises increasing a dose of the immunotherapy.

In some embodiments, at least one time point is at or before initiation of treatment. In some embodiments, at least one time point is after initiation of treatment. In some embodiments, the first time point is before therapy. In some embodiments, the first time point is at initiation of therapy. In some embodiments, the first time point is during remission. In some embodiments, the first time point is before initiation of immunotherapy. In some embodiments, the first time point is at initiation of immunotherapy. In some embodiments, the first time point is during immunotherapy. In some embodiments, the second time point is the current measuring. In some embodiments, the second time point is a test time point. In some embodiments, the second time point is a time point when the subject is at risk for relapse. In some embodiments, the second time point is after initiation of immunotherapy. In some embodiments, the second time point is during immunotherapy. In some embodiments, the first time point is before initiation of immunotherapy and the second time point is after initiation of immunotherapy. In some embodiments, the first and second time points are after initiation of immunotherapy.

In some embodiments, the first and second time points are separated by at least 1, 2, 3, 4, 5, 6, 7, 8, 9, 10, 11, 12, 13, 14, 15, 16, 17, 18, 19, 20 or 21 days. Each possibility represents a separate embodiment of the invention. In some embodiments, the first and second time points are separated by at least 72 hours. In some embodiments, the first and second time points are separated by at least 5 days. In some embodiments, the first and second time points are separated by at least 7 days. In some embodiments, the first and second time points are separated by at least 1, 2, 3, 4, 5, 6, or 7 weeks. Each possibility represents a separate embodiment of the invention. In some embodiments, the first and second time points are separated by at least 1 week. In some embodiments, the first and second time points are separated by at least 6 weeks. In some embodiments, the first and second time points are separated by at least 7 weeks. In some embodiments, the first and second time points are separated by at most 1, 2, 3, 4, 5, 6, or 7 weeks. Each possibility represents a separate embodiment of the invention. In some embodiments, the first and second time points are separated by at most 6 weeks. In some embodiments, the first and second time points are separated by at most 7 weeks. In some embodiments, the first and second time points are separated by about 6 weeks. In some embodiments, the first and second time points are separated by about 7 weeks. In some embodiments, the first and second time points are separated by about 6-7 weeks. In some embodiments, the first and second time points are separated by 6-7 weeks. In some embodiments, the second time point is at least 7, 6, 5, 4 or 3 weeks after initiation of immunotherapy. Each possibility represents a separate embodiment of the invention. In some embodiments, the second time point is at least 7 weeks after initiation of immunotherapy. In some embodiments, the second time point is at least 4 weeks after initiation of immunotherapy. In some embodiments, the first time point is at initiation of immunotherapy. In some embodiments, the first time point is just before initiation of immunotherapy. In some embodiments, just before is not more than 6, 8, 12, 24, 26, 26, 72, 96 or 120 hours before initiation. Each possibility represents a separate embodiment of the invention. In some embodiments, the first time point is 7 weeks after initiation of immunotherapy. In some embodiments, the second time point is 13 weeks after initiation of immunotherapy. In some embodiments, the second time point is 7 weeks or 13 weeks after initiation of immunotherapy.

In some embodiments, the increase is at least a 10, 20, 25, 30, 40, 50, 60, 70, 80, 90, 100, 150, 200, 250, 300, 350, 400, 450, 500, 600, 700, 800, 900, or 1000% increase. Each possibility represents a separate embodiment of the invention. In some embodiments, the increase is at least a 10% increase. In some embodiments, the increase is at least a 25% increase. In some embodiments, the increase is at least a 50% increase. In some embodiments, the increase is at least a 100% increase. In some embodiments, the increase is an increase from no expression of sCD28 to expression. In some embodiments, the increase is from an absence of sCD28 to the presence of sCD28. In some embodiments, the increase is to at least 1, 2, 3, 4, 5, 6, 7, 8, 9 or 10 ng/mL sCD28. Each possibility represents a separate embodiment of the invention. In some embodiments, the increase is to at least 1 ng/mL sCD28. In some embodiments, the increase is to at least 2 ng/mL sCD28. In some embodiments, the increase is to at least 5 ng/mL sCD28. In some embodiments, the increase is to at least 6 ng/mL sCD28. In some embodiments, the increase is an increase of at least 1, 2, 3, 4, 5, 6, 7, 8, 9 or 10 ng/mL sCD28. Each possibility represents a separate embodiment of the invention. In some embodiments, the increase is an increase of at least 1 ng/mL sCD28. In some embodiments, the increase is an increase of at least 2 ng/mL sCD28.

In some embodiments, the predetermined threshold is at least 1, 2, 3, 4, 5, 6, 7, 8, 9 or 10 ng/mL sCD28. Each possibility represents a separate embodiment of the invention. In some embodiments, the predetermined threshold is at least 1 ng/mL sCD28. In some embodiments, the predetermined threshold is at least 2 ng/mL sCD28. In some embodiments, the predetermined threshold is at least 5 ng/mL sCD28.

In some embodiments, the predetermined threshold is an increase of at least 10, 15, 20, 25, 30, 35, 40, 45, 50, 60, 70, 75, 80, 90, 95, 100, 125, 150, 175, 200, 250, 300, 350, 400, 450, 500, 600, 700, 750, 800, 900 or 1000%. Each possibility represents a separate embodiment of the invention. In some embodiments, the predetermined threshold is an increase of at least 10%. In some embodiments, the predetermined threshold is an increase of at least 25%. In some embodiments, the predetermined threshold is an increase of at least 50%. In some embodiments, the predetermined threshold is an increase of at least 100%.

In some embodiments, no change or a decrease indicates the subject is a responder to therapy. In some embodiments, no change or a decrease indicates the subject is responding to therapy. In some embodiments, no change comprises a statistically insignificant change.

In some embodiments, the subject's blood comprises elevated levels of sCD28. In some embodiments, the increase is to elevated levels. In some embodiments, the levels are elevated above those of healthy subjects. In some embodiments, the subject's sCD28 levels are elevated by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1000% above healthy subject levels. Each possibility represents a separate embodiment of the invention. In some embodiments, the levels are elevated above 5, 6, 7, 8, 9, 10, 12, 14, 15, 16, 18, 20, 25, 30, 35, 40, 45 or 50 ng/mL of blood. Each possibility represents a separate embodiment of the invention. In some embodiments, the levels are elevated above 5 ng/mL. In some embodiments, the levels are elevated above 10 ng/mL. In some embodiments, the levels are elevated above 20 ng/mL. In some embodiments, the subject's blood comprises at least 5, 6, 7, 8, 9, 10, 12, 14, 15, 16, 18, 20, 25, 30, 35, 40, 45 or 50 ng sCD28 per mL of blood. Each possibility represents a separate embodiment of the invention. In some embodiments, the subject's blood comprises at least 5 ng/mL sCD28. In some embodiments, the subject's blood comprises at least 10 ng/mL sCD28. In some embodiments, the subject's blood comprises at least 20 ng/mL sCD28.

In some embodiments, the subject's blood comprises healthy levels of sCD28 before relapse. In some embodiments, the subject's comprises non-elevated levels of sCD28 before relapse. In some embodiments, the subject's blood comprises less than 1, 2, 3, 4, 5, 6, 7, 8, 9, 10, 12, 14, 15, 16, 18, 20, 25, 30, 35, 40, 45 or 50 ng/mL sCD28 before relapse. In some embodiments, the subject's blood comprises less than 5ng/mL sCD28 before relapse. In some embodiments, the subject's blood comprises less than 6 ng/mL sCD28 before relapse. Each possibility represents a separate embodiment of the invention. In some embodiments, the subject's blood is the subject's blood before performance of a method of the invention.

In some embodiments, the measuring comprises obtaining a sample from the subject. In some embodiments, the measuring is measuring sCD28 levels in the sample. In some embodiments, the sample is a biopsy. In some embodiments, the sample is a bodily fluid. In some embodiments, the sample is blood. In some embodiments, the sample is from a subject. In some embodiments, the sample comprises a bodily fluid. In some embodiments, the sample comprises tissue. In some embodiments, the sample comprises cells. In some embodiments, the detection is by a secondary antibody. In some embodiments, the detection is with a tagged molecule that binds the sCD28. In some embodiments, the detection is by ELISA. In some embodiments, the detection is by immunohistochemistry. In some embodiments, the detection is by immunoblot.

Agents for detecting sCD28 are described in International Patent Application WO2019/175885, herein incorporated by reference in its entirety.

In some embodiments, measuring comprises measuring at a plurality of time points. In some embodiments, the plurality of time points is at least 2, 3, 4, 5, 6, 7, 8, 9, or 10 time points. Each possibility represents a separate embodiment of the invention. In some embodiments, the plurality of time points is two time point. In some embodiments, the plurality of time points is at least 2 time points. In some embodiments, the plurality of time points comprise a first and a second time point. In some embodiments, at least one time point is at a time when the subject is in remission or cancer free. In some embodiments, at least one time point is at a time when the subject is known to be in remission or cancer free. In some embodiments, a time point when the subject is in remission or cancer free or known to be in remission or cancer free is a time point when the subject has sCD28 levels at or below 1, 2, 3, 4, 5, 6, 7, 8, 9 or 10 ng/mL sCD28. Each possibility represents a separate embodiment of the invention. In some embodiments, the time point is when the subject has sCD28 levels at or below 2 ng/mL. In some embodiments, the time point is when the subject has sCD28 levels at or below 5 ng/mL. In some embodiments, the time point is when the subject has sCD28 levels at or below 6 ng/mL. In some embodiments, at least one time point is after initiation of therapy.

In some embodiments, at least one time point is at a time when the subject is at risk of relapse. In some embodiments, at least one time point is at a time when the subject is suspected of relapse. In some embodiments, at least one time point is at least 1, 2, 3, 4, 5, 6, 7, 8, 9, 10, 15, 20, 25, 30, 35, 40, 45, 50, 55 or 60 weeks after initiation of therapy. Each possibility represents a separate embodiment of the invention. In some embodiments, at least one time point is at least 35 weeks after initiation of therapy. In some embodiments, at least one time point is at least 40 weeks after initiation of therapy. In some embodiments, at least one time point is at least 45 weeks after initiation of therapy. In some embodiments, at least one time point is at least 46 weeks after initiation of therapy.

In some embodiments, a relapse to come is an impending relapse. In some embodiments, impending relapse is imminent relapse. In some embodiments, impending is within the next 10, 15, 20, 25, 30, 35, or 40 weeks. Each possibility represents a separate embodiment of the invention. In some embodiments, a relapse to come is relapse at any time in the future. In some embodiments, imminent relapse is within the next 1, 2, 3, 4, 5, 6, 7, 8, 9, 10, 15 or 20 weeks. Each possibility represents a separate embodiment of the invention.

In some embodiments, the method further comprises administering the immunotherapy to the subject. In some embodiments, the method further comprises administering the immunotherapy between the first and second time points. In some embodiments, the method further comprises administering a treatment that comprises an agent that reduces sCD28 levels. In some embodiments, the treatment that comprises an agent that reduces sCD28 levels comprises performing a method of the invention. In some embodiments, the method comprises administering the agent that reduces sCD28 levels. In some embodiments, administering the agent that reduces sCD28 levels converts a not responding subject to a responding subject. In some embodiments, administering the agent that reduces sCD28 levels converts a non-responder to a responder. In some embodiments, administering the agent that reduces sCD28 levels converts a subject at risk of relapse to one not at risk of relapse. In some embodiments, administering the agent that reduces sCD28 levels converts a subject in relapse to a subject that is responding.

In some embodiments, the another therapy is a therapy that treats the cancer. In some embodiments, the method further comprises administering another therapy to a subject diagnosed with or predicted for a cancer relapse. In some embodiments, the method further comprises administering another therapy to a subject diagnosed with a cancer relapse. In some embodiments, the method further comprises administering another therapy to a subject predicted for a cancer relapse. In some embodiments, the method further comprises administering another therapy to a subject determined to be a non-responder. In some embodiments, the method further comprises administering another therapy to a subject determined to be a non-responder to the immunotherapy. In some embodiments, the other therapy is not the therapy that caused the remission. In some embodiments, the other therapy is a different therapy than the therapy initially used to treat the cancer. In some embodiments, the other therapy is not a PD-1/PD-L1 based immunotherapy. In some embodiments, the other therapy is not an immunotherapy. In some embodiments, the other therapy is not the therapy to which the subject is a non-responder.

In some embodiments, the another therapy is an immunotherapy. In some embodiments, the another therapy is a therapy that treats the cancer. In some embodiments, the another therapy is a PD-1 and/or PD-L1 based immunotherapy. In some embodiments, the another immunotherapy is a checkpoint inhibitor. In some embodiments, the checkpoint inhibitor is a PD-1 and/or PD-L1 inhibitor. In some embodiments, the checkpoint inhibitor is a CTLA-4 inhibitor. In some embodiments, the another immunotherapy is a chimeric antigen receptor (CAR) based immunotherapy. In some embodiments, the CAR is a CAR-T. In some embodiments, the CAR is a CAR-NK. In some embodiments, the another immunotherapy is a cancer vaccine.

In some embodiments, the another therapy is a therapy that reduces sCD28 levels in the subject. In some embodiments, the another therapy comprises administering an agent that reduces sCD28 levels in the subject. In some embodiments, the another therapy administering an agent that reduces sCD28 levels in the subject. In some embodiments, an immunotherapy is an agent that reduces sCD28 levels in the subject.

In some embodiments, an agent that reduces sCD28 levels is an agent that binds sCD28. In some embodiments, upon binding the agent degrades the sCD28. In some embodiments, upon binding the agent target the sCD28 for degradation.

In some embodiments, the agent is not a CD28 agonist. In some embodiments, the agent is not a CD28 antagonist. In some embodiments, the agent is neither a CD28 agonist or antagonist. In some embodiments, the sCD28 binding agent is also an mCD28 agonist.

The term “agonist” generally refers to a molecule, compound or agent that binds to a receptor and activates, fully or partially, the receptor. In some embodiments, the agonist binds at the same site as the natural ligand. In some embodiments, the agonist binds at an allosteric site different from the binding site of the natural ligand. The term “antagonist” generally refers to a molecule, compound or agent that binds to a receptor at the same site as an agonist or another site, does not activate the receptor and does one or more of the following: interferes with or blocks activation of the receptor by a natural ligand, and interferes with or blocks activation of the receptor by a receptor agonist. In some embodiments, the antibodies of the invention bind to mCD28 but do not activate or block activation of the receptor. In some embodiments, they do not block activation by CD86. In some embodiments, the antibodies of the invention do not bind mCD28.

As used herein, a “direct agonist/antagonist” refers to a molecule that binds to a receptor (mCD28) and by binding increases/decreases signaling by that molecule. In the case of mCD28 an agonist would bind mCD28 and by binding increase mCD28 signaling in the cell. In some embodiments, the agonist increases T cell activation. In some embodiments, the agonist increases T cell proliferation. In some embodiments, the agonist increases pro-inflammatory cytokine secretion. Pro-inflammatory cytokines are well known in the art and are known to be secreted by activated T cells. Examples of pro-inflammatory cytokines include, but are not limited to, TNFα, IFNγ, IL-1B, and IL-6. In some embodiments, the pro-inflammatory cytokine is IFNγ. In the case of mCD28 an antagonist would bind mCD28 and by binding decrease mCD28 signaling in the cell. In some embodiments, the antagonist decreases T cell activation, decreases T cell proliferation and/or decreases pro-inflammatory cytokine secretion. A molecule that effects a receptor's signaling by contacting its ligand, contacting an inhibitor, contacting a co-receptor or contacting any molecule other than the receptor in question in order to modify receptor signaling is not considered a direct agonist/antagonist. In some embodiments, the agent of the invention contacts sCD28 in serum and thereby allows for increased signaling through mCD28 on cells. Though the result is increased mCD28 signaling the antibody is not a mCD28 agonist or direct agonist as its binding to mCD28 does not increase the receptors signaling.

In some embodiments, the agent does not bind the ligand binding domain of mCD28. In some embodiments, the agent does not obscure or block access to the ligand binding domain. In some embodiments, the agent does not bind, obscure or block access to the IgV domain of sCD28. In some embodiments, the IgV domain is the ligand binding domain. In some embodiments, the ligand binding domain comprises amino acids 28-137 of SEQ ID NO: 1. In some embodiments, the ligand binding domain comprises or consists of the amino acid sequence MLVAYDNAVNLSCKYSYNLFSREFRASLHKGLDSAVEVCVVYGNYSQQLQVYSKTG FNCDGKLGNESVTFYLQNLYVNQTDIYFCKIEVMYPPPYLDNEKSNGTIIHVKG (SEQ ID NO: 7). In some embodiments, the agent does not inhibit binding of sCD28 to a ligand. In some embodiments, the CD28 ligand is selected from: CD80, CD86 and ICOSL. In some embodiments, the CD28 ligand is CD86. In some embodiments, the CD28 ligand is CD80. In some embodiments, the CD28 ligand is ICOSL. In some embodiments, CD86 is CD86-Fc. In some embodiments, CD80 is CD80-Fc.

An example of an agent includes, but is not limited to, an antibody, an antigen binding fragment of an antibody, a nanobody, a single chain antibody, a single domain antibody, a small molecule, a peptide and a DARPin. In some embodiments, the agent is selected from an antibody, an antigen binding fragment of an antibody, a Fab fragment, a nanobody, a single chain antibody, a single domain antibody, a small molecule, a peptide and a DARPin. In some embodiments, the agent is selected from an antibody, an antigen binding fragment of an antibody, a Fab fragment, a single chain antibody, a single domain antibody, a small molecule, and a peptide with specific binding to CD28. In some embodiments, the agent is a single domain antibody. In some embodiments, the agent is a nanobody. In some embodiments, the agent is a VHH antibody. As used herein, the terms “single domain antibody”, “nanobody” and “VHH antibody” are synonymous and used interchangeably. In some embodiments, the peptide has specific binding to CD28. In some embodiments, the agent is a peptide with specific binding to CD28. In some embodiments, the peptide is selected from an antibody, an antigen binding fragment of an antibody, a Fab fragment, a single chain antibody, a single-domain antibody, a nanobody, a VHH antibody and an antibody mimetic. As used herein, the term “antibody mimetic” refers to an organic compound that can specifically bind to a target antigen. In some embodiments, an antibody mimetic is not structurally related to an antibody. Examples of antibody mimetics include, but are not limited to, affilins, affimers, affitins, alphabodies, anticalins, avimers, DARPins, fynomers, Kunitz domain peptides, monobodies, and nanoCLAMPS. In some embodiments, the antibody mimetic is a DARPin. All of these agents are well known in the art and are known to be useful in blocking interactions between receptors and their ligands. Small molecules and proteins that can bind mCD28 may occlude the cleavage site or may cause hinderance or impair access for the protease. In some embodiments, the protein is an antibody mimetic. As used herein, the term “DARPin” refers to a designed ankyrin repeat protein. DARPins are genetically engineered antibody mimetic proteins that are generally highly specific for their protein target. Thus, a DARPin for CD28 may be an example of an agent.

In some embodiments, the agent that binds sCD28 is an antibody or antigen binding fragment thereof. In some embodiments, the antibody to sCD28 is a single domain antibody. In some embodiments, the antibody to sCD28 lacks an Fc domain. In some embodiments, the agent that binds sCD28 is an antigen binding domain that lacks an Fc domain. In some embodiments, the agent that binds sCD28 is a single-domain antibody. In some embodiments, the agent that binds sCD28 is a camelid, shark or nanobody. In some embodiments, the antibody or fragment is fused to another protein or fragment of a protein. In some embodiments, the second protein or fragment increases half-life, particularly in serum. In some embodiments, the half-life extending protein is human serum albumin. In some embodiments, the agent is modified by a chemical that produces a modification that enhances half-life. In some embodiments, the modification is PEGylation and the chemical is polyethylene glycol. A skilled artisan will appreciate that any half-life extending protein or chemical agent, or modification known in the art may be used.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi- specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2, single stranded antibody (scFv), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass. In some embodiments, the antibody comprises IgG2 or IgG4. In some embodiments, the antibody comprises IgG2. In some embodiments, the antibody comprises IgG4.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e. CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.).

Immunoglobulin variable domains can also be analyzed using the IMGT information system (www://imgt. cines.fr/) (IMGT®/V-Quest) to identify variable region segments, including CDRs. See, e.g., Brochet, X. et al, Nucl. Acids Res. J6:W503-508 (2008).

Chothia et al. also defined a numbering system for variable domain sequences that is applicable to any antibody. One of ordinary skill in the art can unambiguously assign this system of “Chothia numbering” to any variable domain sequence, without reliance on any experimental data beyond the sequence itself. As used herein, “Chothia numbering” refers to the numbering system set forth by Chothia et al., Journal of Molecular Biology, “Canonical Structures for the Hypervariable regions of immunoglobulins” (1987) and Chothia et al., Nature, “Conformations of Immunoglobulin Hypervariable Regions” (1989).

As used herein, the term “humanized antibody” refers to an antibody from a non-human species whose protein sequences have been modified to increase similarity to human antibodies. A humanized antibody may be produced by production of recombinant DNA coding for the CDRs of the non-human antibody surrounded by sequences that resemble a human antibody. In some embodiments, the humanized antibody is a chimeric antibody. In some embodiments, humanizing comprises insertion of the CDRs of the invention into a human antibody scaffold or backbone. Humanized antibodies are well known in the art and any method of producing them that retains the CDRs of the invention may be employed.

The term “monoclonal antibody” or “mAb” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies and is not to be construed as produced by any specific preparation method. Monoclonal antibodies to be used in accordance with the methods provided herein, may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The mAb of the present invention may be of any immunoglobulin class including IgG, IgM, IgD, IgE or IgA. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multispecific antibodies formed from antibody fragments (e.g., including but not limited to, Db-Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three surfaces of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, delta, e, gamma, and micro, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore eds., Springer- Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies production is known in the art and is described in Natl. Acad. Sci. USA, 90:6444-6448 (1993).

The term “multispecific antibody” is used in the broadest sense and specifically covers an antibody that has polyepitopic specificity. Such multispecific antibodies include, but are not limited to, an antibody comprising a heavy chain variable domain (VH) and a light chain variable domain (VL), where the VHVL unit has polyepitopic specificity, antibodies having two or more VL and VH domains with each VHVL unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. “Polyepitopic specificity” refers to the ability to specifically bind to two or more different epitopes on the same or different target(s).

The monoclonal antibodies of the invention may be prepared using methods well known in the art. Examples include various techniques, such as those in Kohler, G. and Milstein, C, Nature 256: 495-497 (1975); Kozbor et al, Immunology Today 4: 72 (1983); Cole et al, pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies, one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

In some embodiments, antibodies and portions thereof include: antibodies, fragments of antibodies, Fab and F(ab′)2, single-domain antigen-binding recombinant fragments and natural nanobodies. In some embodiments, the antigen binding fragment is selected from the group consisting of a Fv, Fab, F(ab′)₂, scFv or a scFv2 fragment.

In some embodiments, the present invention provides nucleic acid sequences encoding the antibodies or antigen binding portions of the present invention.

For example, the polynucleotide may encode an entire immunoglobulin molecule chain, such as a light chain or a heavy chain. A complete heavy chain includes not only a heavy chain variable region (VH) but also a heavy chain constant region (CH), which typically will comprise three constant domains: CHL CH2 and CH3; and a “hinge” region. In some situations, the presence of a constant region is desirable.

Other polypeptides which may be encoded by the polynucleotide include antigen-binding antibody fragments such as single domain antibodies (“sdAbs”), Fv, scFv, Fab′ and CHI and CK or CL domain has been excised. As minibodies are smaller than conventional antibodies they should achieve better tissue penetration in clinical/diagnostic use but being bivalent they should retain higher binding affinity than monovalent antibody fragments, such as sdAbs. Accordingly, unless the context dictates otherwise, the term “antibody” as used herein encompasses not only whole antibody molecules, but also antigen-binding antibody fragments of the type discussed above. Each framework region present in the encoded polypeptide may comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

Suitably, the polynucleotides described herein may be isolated and/or purified. In some embodiments, the polynucleotides are isolated polynucleotides.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring”.

In some embodiments, the agent is an antibody or antigen-binding portion thereof, comprising three heavy chain CDRs (CDR-H) and three light chain CDRs (CDR-L), wherein: CDR-H1 comprises the amino acid sequence set forth in SEQ ID NO: 12 (GYTLTNY), CDR-H2 comprises the amino acid sequence as set forth in SEQ ID NO: 13 (NTYTGK), CDR-H3 comprises the amino acid sequence as set forth in SEQ ID NO: 14 (GDANQQFAY), CDR-L1 comprises the amino acid sequence as set forth in SEQ ID NO: 15 (KASQDINSYLS), CDR-L2 comprises the amino acid sequence as set forth in SEQ ID NO: 16 (RANRLVD), and CDR-L3 comprises the amino acid sequence as set forth in SEQ ID NO: 17 (LQYDEFPPT).

In some embodiments, the agent is an antibody or an antigen-binding portion thereof, comprising three heavy chain CDRs (CDR-H) and there light chain CDRs (CDR-L), wherein: CDR-H1 comprises the amino acid sequence set forth in SEQ ID NO: 18 (GYTFTSY), CDR-H2 comprises the amino acid sequence as set forth in SEQ ID NO: 19 (YPGDGD), CDR-H3 comprises the amino acid sequence as set forth in SEQ ID NO: 20 (NYRYSSFGY), CDR-L1 comprises the amino acid sequence as set forth in SEQ ID NO: 21 (KSSQSLLNSGNQKNYLT), CDR-L2 comprises the amino acid sequence as set forth in SEQ ID NO: 22 (WASTRES), and CDR-L3 comprises the amino acid sequence as set forth in SEQ ID NO: 23 (QSDYSYPLT).

In some embodiments, the agent is an antibody or an antigen-binding portion thereof, comprising three heavy chain CDRs (CDR-H) and there light chain CDRs (CDR-L), wherein: CDR-H1 comprises the amino acid sequence set forth in SEQ ID NO: 24 (GYTFTDY), CDR-H2 comprises the amino acid sequence as set forth in SEQ ID NO: 25 (NPNYDS), CDR-H3 comprises the amino acid sequence as set forth in SEQ ID NO: 26 (SSPYYDSNHFDY), CDR-L1 comprises the amino acid sequence as set forth in SEQ ID NO: 27 (SARSSINYMH), CDR-L2 comprises the amino acid sequence as set forth in SEQ ID NO: 28 (DTSKLAS), and CDR-L3 comprises the amino acid sequence as set forth in SEQ ID NO: 29 (HQRNSYPFT).

By another aspect, there is provided an antibody or antigen antigen-binding portion thereof, comprising three heavy chain CDRs (CDR-H) and three light chain CDRs (CDR-L), wherein: CDR-H1 comprises the amino acid sequence set forth in SEQ ID NO: 12 (GYTLTNY), CDR-H2 comprises the amino acid sequence as set forth in SEQ ID NO: 13 (NTYTGK), CDR-H3 comprises the amino acid sequence as set forth in SEQ ID NO: 14 (GDANQQFAY), CDR-L1 comprises the amino acid sequence as set forth in SEQ ID NO: 15 (KASQDINSYLS), CDR-L2 comprises the amino acid sequence as set forth in SEQ ID NO: 16 (RANRLVD), and CDR-L3 comprises the amino acid sequence as set forth in SEQ ID NO: 17 (LQYDEFPPT).

By another aspect, there is provided an antibody or an antigen-binding portion thereof, comprising three heavy chain CDRs (CDR-H) and there light chain CDRs (CDR-L), wherein: CDR-H1 comprises the amino acid sequence set forth in SEQ ID NO: 18 (GYTFTSY), CDR-H2 comprises the amino acid sequence as set forth in SEQ ID NO: 19 (YPGDGD), CDR-H3 comprises the amino acid sequence as set forth in SEQ ID NO: 20 (NYRYSSFGY), CDR-L1 comprises the amino acid sequence as set forth in SEQ ID NO: 21 (KSSQSLLNSGNQKNYLT), CDR-L2 comprises the amino acid sequence as set forth in SEQ ID NO: 22 (WASTRES), and CDR-L3 comprises the amino acid sequence as set forth in SEQ ID NO: 23 (QSDYSYPLT).

By another aspect, there is provided an antibody or an antigen-binding portion thereof, comprising three heavy chain CDRs (CDR-H) and there light chain CDRs (CDR-L), wherein: CDR-H1 comprises the amino acid sequence set forth in SEQ ID NO: 24 (GYTFTDY), CDR-H2 comprises the amino acid sequence as set forth in SEQ ID NO: 25 (NPNYDS), CDR-H3 comprises the amino acid sequence as set forth in SEQ ID NO: 26 (SSPYYDSNHFDY), CDR-L1 comprises the amino acid sequence as set forth in SEQ ID NO: 27 (SARSSINYMH), CDR-L2 comprises the amino acid sequence as set forth in SEQ ID NO: 28 (DTSKLAS), and CDR-L3 comprises the amino acid sequence as set forth in SEQ ID NO: 29 (HQRNSYPFT).

In some embodiments, the antibody or antigen-binding fragment thereof comprises a heavy chain comprising the amino acid sequence QIQLVQSGPELKKPGETVKISCKASGYTLTNYGMNWVKQAPGKGLKWMGWINTYTG KPTYVDDFKGRFAF SLETSASTAYLQINNLKNEDTATYFCARGDANQQFAYWGQGTL VTVS (SEQ ID NO: 41). In some embodiments, the variable region of the heavy chain comprises and/or consists of SEQ ID NO: 41. In some embodiments, the antibody or antigen-binding fragment thereof comprises a heavy chain comprising the amino acid sequence QIQLVQSGPELKKPGETVKISCKASGYTLTNYGMNWVKQAPGKGLKWMGWINTYTG KPTYVDDFKGRFAF SLETSASTAYLQINNLKNEDTATYFCARGDANQQFAYWGQGTL VTVSAAKTTPPSVYPLAPGSAAQTNSMVTLGCLVKGYFPEPVTVTWNSGSLS SGVHTF PAVLQSDLYTLS S SVTVPS STWPSETVTCNVAHPASSTKVDKKIVPRDCGCKPCICTVP EVSSVFIFPPKPKDVLTITLTPKVTCVVVDISKDDPEVQF SWFVDDVEVHTAQTQPREE QFNSTFRSVSELPIMHQDWLNGKEFKCRVNSAAFPAPIEKTISKTKGRPKAPQVYTIPPP KEQMAKDKVSLTCMITDFFPEDITVEWQWNGQPAENYKNTQPIMDTDGSYFVYSKLN VQKSNWEAGNTFTCSVLHEGLHNHHTEKSLSHSPGK (SEQ ID NO: 42). In some embodiments, the heavy chain consists of SEQ ID NO: 42. Antibody #1, as referred to in this application, was sequenced and found to have a heavy chain consisting of SEQ ID NO: 42. The CDRs of this heavy chain, as determined using Chothia scheme, are SEQ ID NOs: 12-14.

In some embodiments, the antibody or antigen-binding fragment thereof comprises a light chain comprising the amino acid sequence DIKMTQSPSSMYASLGERVTITCKASQDINSYLSWFQQKPGKSPKTLIYRANRLVDGVP SRFSGSGSGQDYSLTISSLEYDDMGIYYCLQYDEFPPTFGAGTKLELK (SEQ ID NO: 43). In some embodiments, the variable region of the light chain comprises and/or consists of SEQ ID NO: 43. In some embodiments, the antibody or antigen-binding fragment thereof comprises a light chain comprising the amino acid sequence DIKMTQSPSSMYASLGERVTITCKASQDINSYLSWFQQKPGKSPKTLIYRANRLVDGVP SRFSGSGSGQDYSLTISSLEYDDMGIYYCLQYDEFPPTFGAGTKLELKRADAAPTVSIFP PSSEQLTSGGASVVCFLNNFYPKDINVKWKIDGSERQNGVLNSWTDQDSKDSTYSMSS TLTLTKDEYERHNSYTCEATHKTSTSPIVKSFNRNEC (SEQ ID NO: 44). In some embodiments, the light chain consists of SEQ ID NO: 44. Antibody #1, as referred to in this application, was sequenced and found to have a light chain consisting of SEQ ID NO: 44. The CDRs of this light chain, as determined using Chothia scheme, are SEQ ID NOs: 15-17. In some embodiments, the antibody heavy chain comprises SEQ ID NO: 41 and the antibody light chain comprises SEQ ID NO: 43. In some embodiments, the antibody heavy chain comprises or consists of SEQ ID NO: 42 and the antibody light chain comprises or consists of SEQ ID NO: 44.

In some embodiments, the antibody or antigen-binding fragment thereof comprises a heavy chain comprising the amino acid sequence QVQLQQ SGAELARPGASVKLSCKASGYTFTSYWMQWIKKRPGQGLEWIGAIYPGDGD TRYTQKFKGKATLTADKS STTAYMQLSSLASEDSAVYFCARNYRYSSFGYWGQGTLV TVSA (SEQ ID NO: 45). In some embodiments, the variable region of the heavy chain comprises and/or consists of SEQ ID NO: 45. In some embodiments, the antibody or antigen-binding fragment thereof comprises a heavy chain comprising the amino acid sequence QVQLQQSGAELARPGASVKLSCKASGYTFTSYWMQWIKKRPGQGLEWIGAIYPGDGD TRYTQKFKGKATLTADKS STTAYMQLSSLASEDSAVYFCARNYRYS SFGYWGQGTLV TVSAAKTTPPSVYPLAPGCGDTTGSSVTLGCLVKGYFPESVTVTWNSGSLSSSVHTFPA LLQSGLYTMSSSVTVPSSTWPSQTVTCSVAHPASSTTVDKKLEPSGPISTINPCPPCKEC HKCPAPNLEGGPSVFIFPPNIKDVLMISLTPKVTCVVVDVSEDDPDVQISWFVNNVEVH TAQTQTHREDYNSTIRVVSTLPIQHQDWMSGKEFKCKVNNKDLPSPIERTISKIKGLVR APQVYILPPPAEQLSRKDVSLTCLVVGFNPGDISVEWTSNGHTEENYKDTAPVLDSDGS YFIYSKLNMKTSKWEKTDSFSCNVRHEGLKNYYLKKTISRSPGK (SEQ ID NO: 46). In some embodiments, the heavy chain consists of SEQ ID NO: 46. Antibody #2, as referred to in this application, was sequenced and found to have a light chain consisting of SEQ ID NO: 46. The CDRs of this heavy chain, as determined using Chothia scheme, are SEQ ID NOs: 18-20.

In some embodiments, the antibody or antigen-binding fragment thereof comprises a light chain comprising the amino acid sequence DIVMTQSPSSLTVTAGEKVTLSCKSSQSLLNSGNQKNYLTWYQQKPGQPPQLLIYWAS TRESGVPDRFTGSGSGTDFTLTISSVQAEDLAVYYCQSDYSYPLTFGAGTKLELK (SEQ ID NO: 47). In some embodiments, the variable region of the light chain comprises and/or consists of SEQ ID NO: 47. In some embodiments, the antibody or antigen-binding fragment thereof comprises a light chain comprising the amino acid sequence DIVMTQSPSSLTVTAGEKVTLSCKSSQSLLNSGNQKNYLTWYQQKPGQPPQLLIYWAS TRESGVPDRFTGSGSGTDFTLTISSVQAEDLAVYYCQSDYSYPLTFGAGTKLELKRADA APTVSIFPPSSEQLTSGGASVVCFLNNFYPKDINVKWKIDGSERQNGVLNSWTDQDSKD STYSMSSTLTLTKDEYERHNSYTCEATHKTSTSPIVKSFNRNEC (SEQ ID NO: 48). In some embodiments, the light chain consists of SEQ ID NO: 48. In some embodiments, the light chain consists of SEQ ID NO: 48. Antibody #2, as referred to in this application, was sequenced and found to have a light chain consisting of SEQ ID NO: 48. The CDRs of this light chain, as determined using Chothia scheme, are SEQ ID NOs: 21-23. In some embodiments, the antibody heavy chain comprises SEQ ID NO: 45 and the antibody light chain comprises SEQ ID NO: 47. In some embodiments, the antibody heavy chain comprises or consists of SEQ ID NO: 46 and the antibody light chain comprises or consists of SEQ ID NO: 48.

In some embodiments, the antibody or antigen-binding fragment thereof comprises a heavy chain comprising the amino acid sequence EVQLQQFGAELVKPGASVKISCKASGYTFTDYNMDWVKQSHGKSLEWIGDINPNYDS TAYNQKFMGKATLTVDKSSNTAYMELRSLTSEDTAVYYCARSSPYYDSNHFDYWGQ GTSLTVSS (SEQ ID NO: 49). In some embodiments, the variable region of the heavy chain comprises and/or consists of SEQ ID NO: 49. In some embodiments, the antibody or antigen-binding fragment thereof comprises a heavy chain comprising the amino acid sequence EVQLQQFGAELVKPGASVKISCKASGYTFTDYNMDWVKQSHGKSLEWIGDINPNYDS TAYNQKFMGKATLTVDKSSNTAYMELRSLTSEDTAVYYCARSSPYYDSNHFDYWGQ GTSLTVSSAKTTPPSVYPLAPGSAAQTNSMVTLGCLVKGYFPEPVTVTWNSGSLSSGV HTFPAVLQSDLYTLSSSVTVPSSTWPSETVTCNVAHPASSTKVDKKIVPRDCGCKPCIC TVPEVSSVFIFPPKPKDVLTITLTPKVTCVVVDISKDDPEVQF SWFVDDVEVHTAQTQP REEQFNSTFRSVSELPIMHQDWLNGKEFKCRVNSAAFPAPIEKTISKTKGRPKAPQVYTI PPPKEQMAKDKVSLTCMITDFFPEDITVEWQWNGQPAENYKNTQPIMDTDGSYFVYS KLNVQKSNWEAGNTFTCSVLHEGLHNHHTEKSLSHSPGK (SEQ ID NO: 50). In some embodiments, the heavy chain consists of SEQ ID NO: 50. Antibody #3, as referred to in this application, was sequenced and found to have a heavy chain consisting of SEQ ID NO: 50. The CDRs of this heavy chain, as determined using Chothia scheme, are SEQ ID NOs: 24-26.

In some embodiments, the antibody or antigen-binding fragment thereof comprises a light chain comprising the amino acid sequence

(SEQ ID NO: 51)
QIVLTQSPAIMSASPGEKVTMTCSARSSINYMHWF
QQKPGTSPKRWIYDTSKLASGVPARFSGSGSGTSY
SLTISNMEAEDAATYYCHQRNSYPFTFGSGTKLEI
K.

In some embodiments, the variable region of the light chain comprises and/or consists of SEQ ID NO: 51. In some embodiments, the antibody or antigen-binding fragment thereof comprises a light chain comprising the amino acid sequence QIVLTQSPAIIVISASPGEKVTMTCSARSSINYMIHWFQQKPGTSPKRWIYDTSKLASGVP ARFSGSGSGTSYSLTISNMEAEDAATYYCHQRNSYPFTFGSGTKLEIKRADAAPTVSIFP PSSEQLTSGGASVVCFLNNFYPKDINVKWKIDGSERQNGVLNSWTDQDSKDSTYSMSS TLTLTKDEYERHNSYTCEATHKTSTSPIVKSFNRNEC (SEQ ID NO: 52). In some embodiments, the light chain consists of SEQ ID NO: 52. Antibody #3, as referred to in this application, was sequenced and found to have a light chain consisting of SEQ ID NO: 52. The CDRs of this light chain, as determined using Chothia scheme, are SEQ ID NOs: 27-29. In some embodiments, the antibody heavy chain comprises SEQ ID NO: 49 and the antibody light chain comprises SEQ ID NO: 51. In some embodiments, the antibody heavy chain comprises or consists of SEQ ID NO: 50 and the antibody light chain comprises or consists of SEQ ID NO: 52.

In some embodiments, the antibody or antigen binding fragment thereof is an anti-CD28 antibody. In some embodiments, the antibody or antigen binding fragment thereof is an anti-sCD28 antibody. An “anti-CD28 antibody”, “an antibody which recognizes CD28”, or “an antibody against CD28” is an antibody that binds CD28, with sufficient affinity and specificity. In some embodiments, the antibody has increased binding to CD28 or sCD28. In some embodiments, the antibody has increased binding to sCD28 as compared to membranal mCD28. In some embodiments, the antibody has increased binding to sCD28 as compared to a commercially available CD28 antibody. In some embodiments, the commercially available CD28 antibody is CD28.2. In some embodiments, the antibody or antigen-binding fragment thereof has specific binding affinity for CD28 or sCD28.

As used herein, the terms “increased binding affinity” and “greater binding affinity” are interchangeable. In some embodiments, antibody or antigen-binding portion thereof of the present invention has a greater binding affinity to sCD28 compared to the mCD28. In one embodiment, greater affinity as used herein is by 10%. In one embodiment, greater affinity as used herein is by 30%. In one embodiment, greater affinity as used herein is by 50%. In one embodiment, greater affinity as used herein is by 75%. In one embodiment, greater affinity as used herein is by 100%. In one embodiment, greater affinity as used herein is by 150%. In one embodiment, greater affinity as used herein is by 250%. In one embodiment, greater affinity as used herein is by 500%. In one embodiment, greater affinity as used herein is by 1,000%. In one embodiment, greater affinity as used herein is by 1.5-fold. In one embodiment, greater affinity as used herein is by 2-fold. In one embodiment, greater affinity as used herein is by 5-fold. In one embodiment, greater affinity as used herein is by 10-fold. In one embodiment, greater affinity as used herein is by 50-fold. In one embodiment, greater affinity as used herein is by 100-fold. In one embodiment, greater affinity as used herein is by 500-fold. In one embodiment, greater affinity as used herein is by 1,000-fold.

An “antigen” is a molecule or a portion of a molecule capable of eliciting antibody formation and being bound by an antibody. An antigen may have one or more than one epitope. The specific reaction referred to above is meant to indicate that the antigen will react, in a highly selective manner, with its corresponding antibody and not with the multitude of other antibodies which may be evoked by other antigens.

The term “antigenic determinant” or “epitope” according to the invention refers to the region of an antigen molecule that specifically reacts with particular antibody. Peptide sequences derived from an epitope can be used, alone or in conjunction with a carrier moiety, applying methods known in the art, to immunize animals and to produce additional polyclonal or monoclonal antibodies.

In some embodiments, the agent is an antibody or antigen binding fragment thereof. In some embodiments, the antigen binding fragment is a Fab fragment. In some embodiments, the antibody is a single domain antibody. In some embodiments, the antibody lacks a Fc domain. In some embodiments, the agent is an antigen binding domain that lacks an Fc domain. In some embodiments, the agent is a single-domain antibody. In some embodiments, the agent is a camelid or shark antibody. In some embodiments, the agent is a nanobody. In some embodiments, the agent is a VHH antibody. In some embodiments, the antibody or fragment is fused to another protein or fragment of a protein. In some embodiments, the second protein or fragment increases half-life, particularly in serum. In some embodiments, the half-life extending protein is human serum albumin. In some embodiments, the agent is modified by a chemical that produces a modification that enhances half-life. In some embodiments, the modification is PEGylation and the chemical is polyethylene glycol. A skilled artisan will appreciate that any half-life extending protein or chemical agent, or modification known in the art may be used.

In some embodiments, binding of the agent to sCD28 degrades sCD28. In some embodiments, the binding of the agent to sCD28 leads to or results in degradation of sCD28. In some embodiments, the degradation occurs when the binding is within an organism. In some embodiments, the degradation occurs when the binding is in the bloodstream of an organism. In some embodiments, the degradation occurs when the binding is in the TME of an organism. In some embodiments, degradation comprises removal of sCD28 from blood. In some embodiments, the degradation comprises transport of the bound sCD28 to a lysosome, endosome, proteasome or a combination thereof. Each possibility represents a separate embodiment of the invention.

In some embodiments, binding of the agent to sCD28 in an organism results in removal of sCD28 from blood. In some embodiments, binding of the agent to sCD28 in an organism results in sweeping of the sCD28 from blood. In some embodiments, binding of the agent to sCD28 in an organism results in removal of sCD28 from the TME. In some embodiments, binding of the agent to sCD28 in an organism results in sweeping of the sCD28 from the TME. In some embodiments, binding of the agent to sCD28 in an organism results in removal of sCD28 from blood, the TME or both. In some embodiments, binding of the agent to sCD28 in an organism results in sweeping of the sCD28 from blood, the TME or both. In some embodiments, the sCD28 is not degraded, but is removed from blood, the TME or both. In some embodiments, the bound sCD28 is an immune complex.

There are many known mechanisms that remove bound antigens from the bloodstream. These include but are not limited to complement mediated removal, phagocytosis by monocytes and macrophages, opsonization, proteolysis, passive diffusion, and active transport. In some embodiments, the active transport is by red blood cells. In some embodiments, the bound antigen is transported to a phagocyte. In some embodiments, the bound antigen is transported to a lysosome. In some embodiments, the bound antigen is transported to a lysosome, endosome, proteasome, or a combination thereof. Any agent that can induce removal of the bound sCD28 complex can be used for the invention.

In some embodiments, the agent reduces sCD28 levels by at least 5, 10, 15, 20, 25, 30, 35, 40, 45, 50, 55, 60, 65, 70, 75, 80, 85, 90, 95, 97 or 99%. Each possibility represents a separate embodiment of the invention. In some embodiments, the agent reduces sCD28 levels to that of a healthy individual. In some embodiments, the agent reduces sCD28 levels to at most 1, 2, 3, 4, 5, 10, 15, 20, 25, 30, 35, 40 ,45, or 50 ng/ml. Each possibility represents a separate embodiment of the invention. In some embodiments, the agent reduces sCD28 blood levels to at most 5 ng/ml. In some embodiments, the agent reduces sCD28 blood levels to at most 10 ng/ml. In some embodiments, the agent reduces sCD28 blood levels to at most 20 ng/ml. In some embodiments, the agent reduces sCD28 levels to that of a healthy individual. In some embodiments, the agent reduces sCD28 levels to below 1, 2, 3, 4, 5, 10, 15, 20, 25, 30, 35, 40 ,45, or 50 ng/ml. Each possibility represents a separate embodiment of the invention. In some embodiments, the agent reduces sCD28 levels to below 5 ng/ml. In some embodiments, the agent reduces sCD28 levels to below 10 ng/ml. In some embodiments, the agent reduces sCD28 levels to below 20 ng/ml. In some embodiments, the reducing or decreasing occurs in blood, peripheral blood or the TME of the subject. In some embodiments, the reducing or decreasing occurs in blood.

In some embodiments, sCD28 levels are as measured by ELISA. In some embodiments, the ELISA is a sandwich ELISA. In some embodiments, the ELISA is a standardized sandwich ELISA. In some embodiments, the ELISA is a Bender MedSystems ELISA. In some embodiments, the ELISA is Bender MedSystems ELISA kit BMS290. In some embodiments, the ELISA is R&D Systems ELISA kit No. 15061. In some embodiments, the ELISA is performed with an agent of the invention.

In some embodiments, the agent is a sweeping antibody. As used herein, the term “sweeping antibody” refers to any antibody or antigen binding fragment thereof that decreases the amount of a soluble component from a solution. In some embodiments the sweeping antibody does not induce antibody dependent cell-mediated cytotoxicity (ADCC). In some embodiments, the sweeping antibody does not induce complement-dependent cytotoxicity (CDC). In some embodiments, the sweeping antibody does not induce ADCC and/or CDC. In some embodiments, the sweeping antibody comprises an IgG2 or IgG4 domain. In some embodiments, the sweeping antibody comprises an IgG2 domain. In some embodiments, the sweeping antibody comprises an IgG4 domain. In some embodiments, the sweeping antibody comprises an IgG1 or IgG3 mutated to reduce cell death mediated by binding of the antibody. In some embodiments, the mutation mutates a Fc receptor binding domain. In some embodiments, a Fc domain of the antibody is engineered or mutated to decrease CDC, ADCC or both. Fc engineering is well known in the art, and any mutation or amino acid change that is known to decrease antibody mediated cell killing may be used.

In some embodiments, the antibody does not comprise IgG1 and/or IgG3. In some embodiments, the antibody does not induce antibody-dependent cell-mediated cytotoxicity (ADCC). In some embodiments the antibody does not induce complement-dependent cytotoxicity (CDC). In some embodiments, the antibody comprises an IgG1 or IgG3 comprising a mutation that reduces ADCC, CDC or both induced by the antibody's binding. In some embodiments, the mutation reduces the ADCC, CDC or both to nothing. ADCC and CDC are well characterized and antibody sequences that allow for these cytotoxic pathways to be induced are well known. Mutations, such as for non-limiting examples, mutation of IgG1 or IgG3 to IgG2 or IgG4 are well known. Any such mutation may be used in the backbone of the antibodies of the invention.

In some embodiments, the Fc domain of the antibody is a human Fc domain. In some embodiments, the Fc domain comprises a mutation that reduces the ADCC, CDC or both. In some embodiments, the antibody comprises a mutation that increases dissociation of the antibody or antigen binding portion thereof from sCD28 at low pH. In some embodiments, the low pH is a pH at or below 6.9, 6.8. 6.7, 6.6, 6.5, 6.4, 6.3, 6.2, 6.1, 6, 5.9, 5.8, 5.7, 5.6, 5.5, 5.4, 5.3, 5.2, 5.1, 5, 4.5, 4, 3.5 or 3. Each possibility represents a separate embodiment of the invention. In some embodiments, low pH is at or below a pH of 6. In some embodiments, low pH is the pH found in a human endosome. In some embodiments, low pH is the pH found in a human lysosome. In some embodiments, the antibody comprises a mutation that increases dissociation of the antibody or antigen binding portion thereof from sCD28 at low calcium concentration. In some embodiments, the low calcium concentration is the calcium concentration at or below 1.2, 1.1, 1.0, 0.9, 0.8, 0.7, 0.6, 0.5, 0.4, 0.3, 0.2, 0.1, 0.07, 0.05, 0.03, or 0.01 mM. Each possibility represents a separate embodiment of the invention. In some embodiments, the low calcium concentration is the calcium concentration found in a human endosome. In some embodiments, the mutation that increases dissociation is a mutation in a CDR. In some embodiments, the antibody comprises a mutation that increased dissociation of the antibody from sCD28 in the endosome and/or lysosome.

In some embodiments, the mutation is in the FcRn binding region. In some embodiments, the mutation is in the FcγRIIb binding region. In some embodiments, the mutation increases binding to a Fc receptor. In some embodiments, the Fc receptor is FcRn. In some embodiments, the Fc receptor is FcyRIIb. In some embodiments, the mutation increases Fc receptor binding at neutral pH and/or in serum. In some embodiments, the mutation increases Fc receptor binding at low pH. In some embodiments, the mutation is selected from mutation to a histidine of amino acid 27 of the heavy chain, amino acid 31 of the heavy chain, amino acid 32 of the light chain and amino acid 53 of the light chain. In some embodiments, the mutation increases uptake into cells of the antibody and its bound antigen.

Examples of mutations that increase Fc receptor binding, increase uptake into a cell, increase dissociation at low pH, and/or are useful in generating sweeping antibodies, are well known in the art. Examples of such can be found in, for example, Schroter et al., A generic approach to engineer antibody pH-switches using combinatorial histidine scanning libraries and yeast display, mAbs, 2015; Yang et al., Maximizing in vivo target clearance by design of pH-dependent target binding antibodies with altered affinity to FcRn, mAbs, 2017; and Igawa et al., Sweeping antibody as a novel therapeutic antibody modality capable of eliminating soluble antigens from circulation, Immunological Reviews, 2016, all of which are incorporated herein by reference in their entirety. Methods of generating such antibodies as well of testing their efficacy are also provided therein. In some embodiments, the mutation is of the human Fc domain and selected from mutation of histidine 268 to glutamine, valine 309 to leucine, alanine 330 to serine and proline 331 to serine. The numbering provided is for IgG1, but comparable mutations, such as can be determined by a skilled artisan, may be made in other IgGs. Further, the efficacy of such antibodies can be examined by performing binding/dissociate assays in media of various pHs. Tests in model organisms such as mice and monkeys are also possible, in which serum levels of sCD28 are measured before and after addition of the antibody. Optionally, tumors expressing human sCD28 may be xenografted to the animal to ensure serum sCD28.

In some embodiments, the agent that reduces sCD28 levels is an agent that binds membranal CD28 (mCD28). In some embodiments, the agent inhibits proteolytic cleavage of the mCD28. In some embodiments, the agent inhibits proteolytic shedding of CD28. In some embodiments, shedding is shedding of the extracellular region of CD28. In some embodiments, proteolytic cleavage is by a matrix metalloprotease (MMP). In some embodiments, the MMP is MMP-2. In some embodiments, the MMP is MMP-13. In some embodiments, the MMP is selected from MMP-2 and MMP-13. In some embodiments, cleavage is cleavage at the MMP cleavage site. In some embodiments, the cleavage site is PX₁X₂/X₃ wherein X₃ is a hydrophobic residue. In some embodiments, the cleavage site is in a stalk domain of CD28. In some embodiments, the stalk region comprises the sequence GKHLCPSPLFPGPSKP (SEQ ID NO: 8). In some embodiments, the stalk region comprises the sequence KGKHLCPSPLFPGPS (SEQ ID NO: 9). In some embodiments, the stalk region comprises or consists of the sequence HVKGKHLCPSPLFPGPSKP (SEQ ID NO: 10). In some embodiments, the cleavage site is PSPL (SEQ ID NO: 11). In some embodiments, the cleavage is between the P and L of PSPL.

As used herein, inhibiting proteolytic cleavage refers to any reduction in proteolytic cleavage of mCD28. In some embodiments, the inhibition is a reduction in cleavage of at least 5, 10, 15, 20, 25, 30, 35, 40, 45, 50, 55, 60, 65, 70, 75, 80, 85, 90, 95, 97, 99 or 100%. Each possibility represents a separate embodiment of the invention. In some embodiments, inhibiting proteolytic cleavage maintains levels of mCD28 on immune cells. In some embodiments, inhibiting proteolytic cleavage increases levels of mCD28 on immune cells. In some embodiments, inhibiting proteolytic cleavage maintains levels of mCD28 adequate for immune-stimulation.

In some embodiments, the agent does not modulate CD28 function and/or signaling. In some embodiments, the agent does not degrade mCD28. In some embodiments, the agent does not lead to or facilitate mCD28 degradation. In some embodiments, the signaling is mCD28-mediated immune cell activation. In some embodiments, the agent does not inhibit immune cell activation. In some embodiments, the agent does not induce CD28 receptor internalization or recycling. Co-stimulation via mCD28 is essential for immune activation of T-cells. Proteolytic cleavage removed the ligand-binding domain in the extracellular region of CD28 from the transmembrane and cytoplasmic portions of the protein which remain in the membrane. Thus, cleaved CD28 cannot signal and cannot contribute to T cell activation. Thus, an agent that blocks cleavage, and is also an antagonist does not allow for mCD28 activation. Similarly, an agent that blocks cleavage, but is also an agonist could induce aberrant T-cell activation, and potentially an autoimmune response.

In some embodiments, an antibody or antigen binding fragment thereof comprises three heavy chain CDRs (CDR-H) and three light chain CDRs (CDR-L), wherein: CDR-H1 comprises the amino acid sequence set forth in SEQ ID NO: 30 (GFTFSSYYMS), CDR-H2 comprises the amino acid sequence as set forth in SEQ ID NO: 31 (TISDGGDNTYYAGTVTG), CDR-H3 comprises the amino acid sequence as set forth in SEQ ID NO: 32 (IHWPYYFDS), CDR-L1 comprises the amino acid sequence as set forth in SEQ ID NO: 33 (RASSSVSYMN), CDR-L2 comprises the amino acid sequence as set forth in SEQ ID NO: 34 (ATSDLAS), and CDR-L3 comprises the amino acid sequence as set forth in SEQ ID NO: 35 (QQWSSHPPT).

In some embodiments, the antibody or antigen-binding fragment thereof comprises a heavy chain comprising the amino acid sequence DVKLVESGGGLVKLGGSLKLSCVASGFTF SSYYMSWVRQTPEKRLEWVATISDGGDN TYYAGTVTGRFTISRDFAKNTLYLQMNSLTSEDTAVYYCARIHWPYYFDSWGQGTTL TVSS (SEQ ID NO: 53). In some embodiments, the variable region of the heavy chain comprises and/or consists of SEQ ID NO: 53. In some embodiments, the antibody or antigen-binding fragment thereof comprises a heavy chain variable region encoded by the nucleotide sequence GACGTGAAGCTCGTGGAGTCTGGGGGAGGCTTAGTGAAGCTTGGAGGGTCCCTGA AACTCTCCTGTGTAGCCTCTGGATTCACTTTCAGTAGCTATTACATGTCTTGGGTTC GCCAGACTCCGGAGAAGAGGCTGGAGTGGGTCGCGACCATAAGTGATGGTGGTGA TAACACCTACTACGCAGGCACTGTGACGGGCCGATTCACCATCTCCAGAGACTTTG CCAAGAACACCCTGTACCTGCAAATGAACAGTCTGACCTCTGAGGACACAGCCGT GTATTACTGTGCAAGAATTCATTGGCCTTACTATTTTGACTCCTGGGGCCAAGGCA CCACTCTCACAGTCTCCTCA (SEQ ID NO: 54). An anti-cleavage antibody of this application, was sequenced and found to have a heavy chain variable region consisting of SEQ ID NO: 53. The CDRs of this heavy chain, as determined using Chothia scheme, are SEQ ID NOs: 30-32.

In some embodiments, the antibody or antigen-binding fragment thereof comprises a light chain comprising the amino acid sequence

(SEQ ID NO: 55)
QFVLSQSPAILSASPGEMLTMTCRASSSVSYMNWY
QQKPGSSPKPWIYATSDLASGVPARFSGSGSGTSY
SLTISRVEAEDAATYYCQQWSSHPPTFGGGTKLEI
R.

In some embodiments, the variable region of the light chain comprises and/or consists of SEQ ID NO: 55. In some embodiments, the antibody or antigen-binding fragment thereof comprises a light chain variable region encoded by the nucleotide sequence CAATTTGTTCTCTCCCAGTCTCCAGCAATCCTGTCTGCATCTCCCGGGGAGATGCTC ACAATGACTTGCAGGGCCAGCTCAAGTGTAAGTTATATGAACTGGTATCAGCAGA AGCCAGGATCTTCCCCCAAACCCTGGATTTATGCCACATCCGACCTGGCTTCTGGA GTCCCTGCTCGCTTCAGTGGCAGTGGGTCTGGGACCTCTTATTCTCTCACAATCAGC AGAGTGGAGGCTGAAGATGCTGCCACTTATTACTGCCAGCAGTGGAGTAGTCACCC ACCCACGTTCGGAGGGGGGACCAAGCTGGAAATAAGA (SEQ ID NO: 56). In some embodiments, the light chain variable region consists of a sequence encoded by SEQ ID NO: 56. An anti-cleavage antibody, as referred to in this application, was sequenced and found to have a light chain variable region consisting of SEQ ID NO: 55. The CDRs of this light chain, as determined using Chothia scheme, are SEQ ID NOs: 33-35. In some embodiments, the antibody comprises a heavy chain variable region comprising or consisting of SEQ ID NO: 53 and a light chain variable region comprising or consisting of SEQ ID NO: 55.

In some embodiments, the agent is an agent provided in International Patent Application WO2019175885, herein incorporated by reference in its entirety.

In some embodiments, the agent is not a full-size antibody. In some embodiments, the agent is not an IgG. In some embodiments, the agent is smaller than 100 kilodaltons (kDa). In some embodiments, the agent is smaller than 100, 95, 90, 85, 80, 75, 70, 65, 60, 55, 50, 45, 40, 35, 30, 25, 20 or 15 kDa. Each possibility represents a separate embodiment of the invention. In some embodiments, the agent is smaller than 50 kDa. In some embodiments, the agent is smaller than 25 kDa. In some embodiments, the agent is smaller than 20 kDa. In some embodiments, the agent is smaller than 15 kDa. In some embodiments, the agent is a camelid antibody. In some embodiments, the agent is a VHH. In some embodiments, the agent is a nanobody.

As used herein, the terms “single chain antibodies” and “single chain variable fragments” are used synonymously and refer to a fusion protein of variable region of heavy and light chains of immunoglobulins, connected by a short peptide linker. In some embodiments a single chain antibody has a size of less than 50, 45, 40, 35, 30, 25, or 20 kDa. Each possibility represents a separate embodiment of the invention. In some embodiments, a single chain antibody has a size of less than 25 kDa. In some embodiments, the linker of a single chain antibody is between 10 and 25 amino acids. In some embodiments, the linker is between 1-40, 5-40, 10-40, 1-35, 5-35, 10-35, 1-30, 5-30, 10-30, 1-25, 5-25 or 10-25 amino acids. Each possibility represents a separate embodiment of the invention. In some embodiments, the, the single chain antibody comprises a heavy chain of antibody M9. In some embodiments, the single chain antibody comprises a light chain of antibody M9. In some embodiments, the single chain antibody comprises the CDRs of antibody M9.

As used herein, the terms “single domain antibody”, “nanobody” and “VHH” are used synonymously and refer to an antibody fragment consisting of a single monomeric variable antibody domain. In some embodiments, the single domain antibody is a camelid antibody. In some embodiments, a camelid is a camel, an alpaca or a llama. In some embodiments, the camelid is a camel. In some embodiments, the camelid is an alpaca. In some embodiments, the camelid is a llama. In some embodiments, the single domain antibody is a shark antibody.

Also, as already indicated herein, the amino acid residues of a Nanobody are numbered according to the general numbering for Vkis given by Kabat et al. (“Sequence of proteins of immunological. interest”, US Public Health Services, NIB Bethesda, Md., Publication No. 91), as applied to VHH domains from Camelids in the article of Riechmann and Muyldermans, Immunol. Methods 2000 Jun. 23; 240 (1-2): 185-195; or referred to herein. According to this numbering, :11Z1 of a Nanobody comprises the amino acid residues at positions 1-30, DRI of a Nanobody comprises the amino acid residues at positions 31-35, FR2 of a Nanobody comprises the amino acids at positions 36-49. CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-1⁻13. in this respect, it should be noted that as is well known in the art for Vii domains and for domains the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.

In some embodiments, the agent comprises three CDRs, wherein CDR1 comprises the amino acid sequence set forth in SEQ ID NO: 57 (INAMG), CDR2 comprises the amino acid sequence as set forth in SEQ ID NO: 58 (AISGGGDTYYADSVKG), CDR3 comprises the amino acid sequence as set forth in SEQ ID NO: 59 (DLYGSDYWD). In some embodiments, the agent comprises three CDRs, wherein CDR1 comprises the amino acid sequence set forth in SEQ ID NO: 36 (INAMA), CDR2 comprises the amino acid sequence as set forth in SEQ ID NO: 37 (AITSSGSTNYANSVKG), CDR3 comprises the amino acid sequence as set forth in SEQ ID NO: 38 (DEYGSDYWI). In some embodiments, the agent comprises three CDRs, wherein CDR1 comprises the amino acid sequence set forth in SEQ ID NO: 57 (INAMG), CDR2 comprises the amino acid sequence as set forth in SEQ ID NO: 39 (AITSGGSTNYADSVKG), CDR3 comprises the amino acid sequence as set forth in SEQ ID NO: 40 (DLYGEDYWI).

In some embodiments, CDR1 comprises the amino acid sequence set forth in SEQ ID NO: 63 (INAMX₁), wherein X₁ is G or A. In some embodiments, CDR2 comprises the amino acid sequence set forth in SEQ ID NO: 64 (AIX₁X₂X₃GX₄TX₅YAX₆SVKG), wherein X₁ is S or T, X₂ is G or S, X₃ is G or S, X₄ is D or S, X₅ is Y or N, and X₆ is D or N. In some embodiments, CDR3 comprises the amino acid sequence set forth in SEQ ID NO: 65 (DX₁YGX₂DYWX₃), wherein X₁ is E or L, X₂ is E or S, and X₃ is D or I. In some embodiments, CDR₃ comprises the amino acid sequence set forth in SEQ ID NO: 66 (DX₁YGSDYWX₂), wherein X₁ is E or L, and X2 is D or I. In some embodiments, the agent comprises no other CDRs other than the CDRs recited hereinabove.

In some embodiments, the agent comprises a sequence comprising and/or consisting of EVQLVESGGGLVQAGESLRLSCAASGSIASINAMGWYRQAPGSQRELVAAISGGGDTY YADSVKGRFTISRDNAKTTVYLQMNSLRPEDTAVYYCVVDLYGSDYWDWGQGTQVT VSS (SEQ ID NO: 60). In some embodiments, the agent comprises a sequence comprising and/or consisting of EVQLVESGGGLVQAGGSLRLSCAASGSLFSINAMAWYRQAPGKQRELVAAITSSGSTN YANSVKGRF TVSRDNAKNTMYLQMNSLKPEDTAVYYCVVDEYGSDYWIWGQGTQV TVSS (SEQ ID NO: 61). In some embodiments, the agent comprises a sequence comprising and/or consisting of QVQLVESGGGLVQAGGSLRLSCAASGSIFSINAMGWYRQAPGKQRERVAAITSGGSTN YADSVKGRFTISRDNAKNTVYLQMNNLEPRDAGVYYCVVDLYGEDYWIWGQGTQVT VSS (SEQ ID NO: 62). In some embodiments, the agent comprises a sequence comprising and/or consisting of EVQLVESGGGLVQAGESLRL SCAASGSIASINSMGWYRQAPGSQRELVAAINEKLLIYY ADSVKGRFTISRDNAKTTVYLQMNSLRPEDTAVYYCVVDLYGSDYWDWGQGTQVTV SS (SEQ ID NO: 92). In some embodiments, the agent comprises a sequence comprising and/or consisting of EVQLVESGGGLVQAGESLRLSCAASGSIASINAMGWYRQAPGSQRELVAAISGGGDTY YADSVKGRF TISRDNAKTTVYLQMNSLRPEDTAVYYCVVDMIEQQWWYWGQGTQV TVSS (SEQ ID NO: 93). In some embodiments, the agent comprises a sequence comprising and/or consisting of

EVQLVESGGGLVQAGESLRLSCAASGSIASINAMGWYRQAPGSQRELVAAISGGGDTY YADSVKGRFTISRDNAKTTVYLQMNSLRPEDTAVYYCVVDTHRGVYWYWGQGTQV TVSS (SEQ ID NO: 94). In some embodiments, the agent comprises a sequence comprising and/or consisting of EVQLVESGGGLVQAGESLRL S CAA S GSIA SIKTMAWYRQAP GS QRELVAAINYIKEIYY ADSVKGRFTISRDNAKTTVYLQMNSLRPEDTAVYYCVVDVTKEDYWYWGQGTQVTV SS (SEQ ID NO: 95). In some embodiments, the agent comprises a sequence comprising and/or consisting of EVQLVESGGGLVQAGESLRL S CAA S GSIA SINSMAWYRQAP GS QRELVAAI SNAREVY YADSVKGRFTISRDNAKTTVYLQMNSLRPEDTAVYYCVVDVYFQEYWYWGQGTQVT VSS (SEQ ID NO: 96). In some embodiments, the agent comprises a sequence comprising and/or consisting of EVQLVESGGGLVQAGESLRL S CAA S GSIA SINTMAWYRQAP GS QRELVAAINSI SRTYY ADSVKGRFTISRDNAKTTVYLQMNSLRPEDTAVYYCVVDVTKEDYWYWGQGTQVTV SS (SEQ ID NO: 97). In some embodiments, the agent comprises a sequence comprising and/or consisting of EVQLVESGGGLVQAGESLRLSCAASGSIASIKTMAWYRQAPGSQRELVTAIASDNRKY YADSVKGRFTISRDNAKTTVYLQMNSLRPEDTAVYYCVVDVTKEDYWYWGQGTQVT VSS (SEQ ID NO: 98). In some embodiments, the agent comprises a sequence comprising and/or consisting of EVQLVESGGGLVQPGGSLRLSCAASGSIASIKTMAWYRQAPGKQRELVTAIASDNRKY YADSVKGRFTISRDNSKNTLYLQMNSLRAEDTAVYYCVVDVTKEDYWYWGQGTLVT VSS (SEQ ID NO: 99). In some embodiments, the agent comprises a sequence comprising and/or consisting of EVQLVESGGGLVQPGGSLRLSCKASGSIASIKTMAWYRQAPGKGLELVTAIASDNRKY YADSVKGRFTISRDNSKTTVYLQMNSLRAEDTAVYYCVVDVTKEDYWYWGQGTLVT VSS (SEQ ID NO: 100). In some embodiments, the agent comprises a sequence comprising and/or consisting of EVQLVESGGGLVQPGGSLRLSCAASGSTASIKTMAWYRQAPGKGLELVTAIASDNRK YYADSVKGRFTISRDNSKTTVYLQMNSLRAEDTAVYYCVVDVTKEDYWYWGQGTLV TVSS (SEQ ID NO: 101). In some embodiments, the agent comprises a sequence comprising and/or consisting of EVQLVESGGGLVQPGGSLRLSCKASGSTASIKTMAWYRQAPGKGLELVTAIASDNRK YYADSVKGRFTISRDNSKTTVYLQMNSLRAEDTAVYYCVVDVTKEDYWYWGQGTLV TVSS (SEQ ID NO: 102). In some embodiments, the agent comprises a sequence comprising and/or consisting of EVQLVESGGGLVQPGGSLRLSCAASGSIASIKTMAWYRQAPGKGRELVTAIASDNRKY YADSVKGRFTISRDNSKTTVYLQMNSLRAEDTAVYYCVVDVTKEDYWYWGQGTLVT VSS (SEQ ID NO: 103). In some embodiments, the agent comprises a sequence comprising and/or consisting of

EVQLVESGGGLVQAGESLRLSCAASGSIASIRTMAWYRQAPGSQRELVAAISSGREVY YADSVKGRFTISRDNAKTTVYLQMNSLRPEDTAVYYCVVDMYWQDYWWWGQGTQ VTVSS (SEQ ID NO: 104). In some embodiments, the agent comprises a sequence comprising and/or consisting of EVQLVESGGGLVQPGESLRLSCAASGSIASIRTMAWYRQAPGSQRELVAAISSGREVY YADSVKGRFTISRDNAKTTVYLQMNSLRAEDTAVYYCVVDMYWQDYWWWGQGTQ VTVSS (SEQ ID NO: 105). In some embodiments, the agent comprises a sequence comprising and/or consisting of EVQLVESGGGLVQPGGSLRLSCKASGSIASIRTMAWYRQAPGKGLELVAAISSGREVY YADSVKGRFTISRDNSKTTVYLQMNSLRAEDTAVYYCVVDMYWQDYWWWGQGTL VTVSS (SEQ ID NO: 106). In some embodiments, the agent comprises a sequence comprising and/or consisting of EVQLVESGGGLVQPGGSLRLSCKASGSTASIRTMAWYRQAPGKGLELVSAISSGREVY YADSVKGRFTISRDNSKTTVYLQMNSLRAEDTAVYYCVVDMYWQDYWWWGQGTL VTVSS (SEQ ID NO: 107). In some embodiments, the agent comprises a sequence comprising and/or consisting of EVQLVESGGGLVQPGGSLRLSCAASGSIASIRTMAWYRQAPGKGLELVSAISSGREVY YADSVKGRFTISRDNSKTTVYLQMNSLRAEDTAVYYCVVDMYWQDYWWWGQGTL VTVSS (SEQ ID NO: 108). In some embodiments, the agent comprises a sequence comprising and/or consisting of

(SEQ ID NO: 109)
EVQLVESGGGLVQAGESLRLSCAASGSIASINSMG
WYRQAPGSQRELVAAISDRSEKYYADSVKGRFTIS
RDNAKTTVYLQMNSLRPEDTAVYYCVVDHHHSDWW
TWGQGTQVTVSS.

In some embodiments, the VHH sequences further comprise a His tag. In some embodiments, the His tag is at least 1, 2, 3, 4, 5, 6, 7, 8, 9, or 10 histidine residues. Each possibility represents a separate embodiment of the invention. In some embodiments, the His tag consists of 6 histidine residues. In some embodiments, the His tag is connected to the VHH via a linker. In some embodiments, the linker is a peptide linker. In some embodiments, the linker is an alanine repeat linker. In some embodiments, the alanine repeat comprises at least 1, 2, 3, 4, 5, 6, 7, 8, 9, or 10 alanine residues. Each possibility represents a separate embodiment of the invention. In some embodiments, the alanine repeat linker consists of 3 alanine residues. In some embodiments, the His-tag is a six His tag.

In some embodiments, the VHH sequences found to specifically bind the stalk region of human CD28 and comprising a His tag are:

(SEQ ID NO: 67, clone 2A1)
EVQLVESGGGLVQAGESLRLSCAASGSIASINAMG
WYRQAPGSQRELVAAISGGGDTYYADSVKGRFTIS
RDNAKTTVYLQMNSLRPEDTAVYYCVVDLYGSDYW
DWGQGTQVTVSSAAAHHHHHH;
(SEQ ID NO: 68, clone 4A4)
YANSVKGRFTVSRDNAKNTMYLQMNSLKPEDTAVY
YCVVDEYGSDYWIWGQGTQVTVSSAAAHHHHHH;
and
(SEQ ID NO: 69, clone 4A1)
QVQLVESGGGLVQAGGSLRLSCAASGSIFSINAMG
WYRQAPGKQRERVAAITSGGSTNYADSVKGRFTIS
RDNAKNTVYLQMNNLEPRDAGVYYCVVDLYGEDYW
IWGQGTQVTVSSAAAHHHHHH.

In some embodiments, the agent is an agent provided in International Patent Application WO2020183473, herein incorporated by reference in its entirety. In some embodiments, the agent is an agent provided in International Patent Application WO2023031943, herein incorporated by reference in its entirety.

In some embodiments, the agent further comprises a moiety that increases stability of the agent. In some embodiments, the moiety that increase stability is a second moiety. In some embodiments, the agent comprises a first moiety that binds CD28. In some embodiments, CD28 is mCD28. In some embodiments, CD28 is sCD28. In some embodiments, the first moiety inhibits proteolytic cleavage. In some embodiments, the first moiety is the agent. In some embodiments, the moiety is attached to the agent by a linker. In some embodiments, the first and second moiety are separated by a linker.

In some embodiments, at least one of the moieties is a binding moiety. In some embodiments, at least one of the moieties is a half-life enhancing moiety. In some embodiments, at least one of the moieties is a half-life extending moiety. In some embodiments, at least one of the moieties is a stability enhancing moiety. In some embodiments, at least one of the moieties is a stability increasing moiety.

In some embodiments, the agent, the first moiety, the second moiety or a combination thereof is not a CD28 agonist. In some embodiments, the agent, the first moiety, the second moiety or a combination thereof is not a CD28 antagonist. In some embodiments, the agent, the first moiety, the second moiety or a combination thereof is neither a CD28 agonist or antagonist.

In some embodiments, the agent comprises a polypeptide. In some embodiments, the polypeptide is fused to a shielding molecule that is not a polypeptide. As used herein, the term “shielding molecule” refers to a moiety that protects the first moiety from degradation, clearance or removal. In some embodiments, the shielding molecule is a polymer. In some embodiments, the polymer is a copolymer. In some embodiments, the polymer is a biodegradable polymer. In some embodiments, the polymer is a polysaccharide polymer. In some embodiments, the polymer is a protein polymer. In some embodiments, the protein polymer is an unstructured protein polymer. Examples of polymers include, but are not limited to, natural polysaccharides, semi-synthetic polysaccharides, O-linked oligosaccharides, N-linked oligosaccharides, dextran, agarose, alginate, chitosan, carrageenan, hydroxyethyl starch (HES), polysialic acid, hyaluronic acid, homo-amino acid polymers, elastin-like polymers, XTEN, PAS, polyethylene glycol (PEG), Poly-(glycolic acid) (PGA) and poly-(lactic acid) (PLA), poly-(lactic-co-glycolic acid) (PLGA) and Poly-D,L-lactic Acid (PDLLA). In some embodiments, the polymer is a biocompatible polymer. In some embodiments, the shielding molecule comprises a polyethylene glycol (PEG) molecule. In some embodiments, the polymer is PEG. In some embodiments, the polymer is selected from PEG, PLGA, PGA, PLA, and PDLLA. In some embodiments, the shielding molecule comprises a PLGA molecule. In some embodiments, the shielding molecule comprises a PGA molecule. In some embodiments, the shielding molecule comprises a PLA molecule. In some embodiments, the shielding molecule comprises a PDLLA molecule. In some embodiments, the shielding molecule comprises an oligosaccharide polymer selected from dextran, agarose, alginate, chitosan, carrageenan, HES, polysialic acid and hyaluronic acid. In some embodiments, the shielding molecule comprises a protein polymer selected from XTEN and PAS. In some embodiments, the shielding molecule comprises a plurality of PEG molecules. In some embodiments, the agent comprises a polypeptide fused to a PEG molecule or to a plurality of PEG molecules. In some embodiments, the agent comprises a polypeptide but does not comprise a PEG molecule. In some embodiments, the agent comprises a polypeptide fused to a polymer molecule or to a plurality of polymer molecules.

In some embodiments, a second moiety comprises a PEG molecule. In some embodiments, a second moiety comprises a plurality of PEG molecules. In some embodiments, the second moiety is polyethylene glycol (PEG). In some embodiments, the second moiety is a polyethylene glycol (PEG) molecule. In some embodiments, the second moiety comprises PEG or a PEG molecule. In some embodiments, the PEG is linear PEG. In some embodiments, the PEG is chained PEG. In some embodiments, the PEG is chains of PEG. In some embodiments, the PEG is branched PEG. In some embodiments, the PEG comprises PEG methyl ether. In some embodiments, the PEG is PEG dimethyl ether.

In some embodiments, the PEG is low molecular weight PEG. In some embodiments, the PEG is high molecular weight PEG. In some embodiments, the PEG comprises a molecular weight of at least 100, 200, 300, 400, 500, 600, 700, 800, 900, 1000, 1500, 2000, 2500, 3000, 3500, 4000, 4500, 5000, 10000, 15000 or 20000 grams/mol. Each possibility represents a separate embodiment of the invention. In some embodiments, the PEG comprises a molecular weight of at most 500, 600, 700, 800, 900, 1000, 1500, 2000, 2500, 3000, 3500, 4000, 4500, 5000, 10000, 15000, 20000, 25000, 30000, 35000, 40000, 45000, or 50000 grams/mol. Each possibility represents a separate embodiment of the invention. In some embodiments, the PEG comprises a molecular weight of about 100, 200, 300, 400, 500, 600, 700, 800, 900, 1000, 1500, 2000, 2500, 3000, 3500, 4000, 4500, 5000, 10000, 15000, 2000, 2500, 3000, 3500, 4000, 4500, 5000, 10000, 15000, 20000, 25000, 30000, 35000, 40000, 45000, or 50000 grams/mol. Each possibility represents a separate embodiment of the invention. In some embodiments, the PEG comprises a molar mass of at least 2 KDa. In some embodiments, the PEG comprises a size of at least 2 KDa. In some embodiments, the PEG comprises a molar mass of between 2 and 40 KDa. In some embodiments, the PEG comprises a size of between 2 and 40 KDa. In some embodiments, a PEG of less than 2 KDa does not produce half-life extension.

In some embodiments, the PEG molecule or molecules is attached to the polypeptide at a carboxylic acid residue. In some embodiments, the PEG molecule or molecules is attached to the polypeptide at a cysteine residue. In some embodiments, the PEG molecule or molecules is attached to the polypeptide at an aspartic acid residue. In some embodiments, the PEG molecule or molecules is attached to the polypeptide at a glutamic acid residue. In some embodiments, the PEG molecule or molecules is attached to the polypeptide at a lysine residue. In some embodiments, the PEG molecule or molecules is attached to the polypeptide at an aspartic acid residue, a glutamic acid residue, a lysine residue or a cysteine residue, each possibility represents a separate embodiment of the invention. In some embodiments, the PEG conjugated to a cysteine via a thiol linkage. In some embodiments, the PEG is conjugated to a C-terminus of the first moiety. In some embodiments, the PEG is conjugated to a C-terminus of the linker. In some embodiments, the PEG is conjugated to an amino acid residue proximal to a C-terminus of the first moiety. In some embodiments, the PEG is conjugated to an amino acid residue proximal to a C-terminus of the linker. In some embodiments, proximal is within 10 amino acids. It will be understood by a skilled artisan that conjugation close to the C-terminus will keep the PEG moiety away from the CDRs and thus decrease the chance of interfering with binding.

As used herein, “PEGylation” is the process of both covalent and non-covalent attachment or amalgamation of PEG to molecules and macrostructures. Methods of PEGylation are well known in the art and are disclosed in for example U.S. Pat. No. 7,610,156, which is incorporated by reference herein.

In some embodiments, the PEG is conjugated directly to the first moiety. In some embodiments, the PEG is conjugated directly to the linker. In some embodiments, the conjugation is an irreversible conjugation. In some embodiments, the PEG is conjugated to a chemical group and the chemical group is bound to the first moiety. In some embodiments, the PEG is conjugated to a chemical group and the chemical group is bound to the linker. In some embodiments, bond between the chemical group and the first moiety or linker is reversible. For example, a PEG substituted with an SPDP group (2-pyridyl-dithio, also known as OPSS-ortho-pyridine disulfide) can react via said group with a cysteine residue to form a reversible disulfide bond. In some embodiments, the PEG is irreversible conjugated. In some embodiments, the PEG is reversibly conjugated.

In some embodiments, the agent comprises a linker. In some embodiments, the at least two moieties are separated by at least one linker. In some embodiments, the at least two moieties are separated by a linker. In some embodiments, the two moieties are separated by a linker. In some embodiments, the two moieties are joined by a linker. In some embodiments, the agent comprises a linker between at least two moieties. In some embodiment, the agent comprises a linker between a first moiety and a second moiety.

In some embodiments, the linker is a peptide linker. In some embodiments, the linker is a flexible linker. In some embodiments, a flexible linker comprises or consists of at least one GGGGS (SEQ ID NO: 70) sequence. In some embodiments, a flexible linker comprises or consists of at least one GGGS (SEQ ID NO: 71) sequence. In some embodiments, a flexible linker comprises or consists of at least one GGGGS repeat. In some embodiments, a flexible linker comprises or consists of 1, 3, or 7 GGGGS repeats. Each possibility represents a separate embodiment of the invention. In some embodiments, a flexible linker comprises or consists of 1, 2, 3, 4, 5, 6, 7, 8, 9, or 10 GGGGS repeats. Each possibility represents a separate embodiment of the invention. In some embodiments, a flexible linker comprises or consists of 1, 2, 3, 4, 5, 6, 7, 8, 9, or 10 GGGS repeats. Each possibility represents a separate embodiment of the invention. In some embodiments, a flexible linker comprises or consists of 1 GGGGS repeat. In some embodiments, a flexible linker comprises or consists of 3 GGGGS repeats. In some embodiments, a flexible linker comprises or consists of 7 GGGGS repeats.

In some embodiments, the linker is of a length sufficient to allow binding of the first moiety to mCD28. In some embodiments, the linker is of a length sufficient to allow binding of the first moiety to its target epitope. In some embodiments, the linker is of a length sufficient to allow binding of the first moiety to the stalk region of mCD28 on a cell. It will be understood by a skilled artisan that the connection of a protective moiety to a small moiety capable of binding the stalk region of mCD28 on the cell surface will need to be of a sufficient length so that the protective moiety does not generate steric hindrance that would perturb the ability of the first moiety to bind. Thus, the linker must be of a sufficient length to allow a range of movement of the first moiety that allows it to access the stalk domain. In some embodiments, the agent does not induce mCD28 crosslinking. In some embodiments, the agent does not induce mCD28 crosslinking that induces immune activation.

In some embodiments, the linker comprises or consists of at least 1, 2, 3, 4, 5, 6, 7, 8, 9, 10, 11, 12, 13, 14, 15, 16, 17, 18, 19, 20, 25, 30, 35, 40, 45 or 50 amino acids. Each possibility represents a separate embodiment of the invention. In some embodiments, the linker comprises or consists of at least 1 amino acid. In some embodiments, the linker comprises or consists of at least 2 amino acids. In some embodiment, the linker is a dipeptide. In some embodiments, the dipeptide is GC. In some embodiments, the linker comprises a cysteine. In some embodiments, the cysteine is a C-terminal cysteine. In some embodiments, the cysteine is proximal to the C-terminus. In some embodiments, proximal is within 50, 45, 40, 35, 30, 25, 20, 15, 10, 5, 3, 2 or 1 amino acid of the C-terminus. Each possibility represents a sperate embodiment of the invention. In some embodiments, proximal is within 10 amino acids of the C-terminus. In some embodiments, the C-terminus is the C-terminus of the first moiety. In some embodiments, the C-terminus is the C-terminus of the linker. In some embodiments, the linker comprises or consists of at least 5 amino acids. In some embodiments, the linker comprises or consists of at least 10 amino acids. In some embodiments, the linker comprises or consists of at least 15 amino acids. In some embodiments, the linker comprises or consists of at least 35 amino acids. In some embodiments, the linker comprises or consists of at most 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 amino acids. Each possibility represents a separate embodiment of the invention. In some embodiments, the linker comprises or consists of at most 5 amino acids. In some embodiments, the linker comprises or consists of at most 15 amino acids. In some embodiments, the linker comprises or consists of at most 20 amino acids. In some embodiments, the linker comprises or consists of at most 30 amino acids. In some embodiments, the linker comprises or consists of at most 35 amino acids. In some embodiments, the linker comprises or consists of at most 40 amino acids. In some embodiments, the linker comprises or consists of at most 50 amino acids. In some embodiments, the linker comprises or consists of between 5-100, 5-75, 5-50, 5-35, 5-15, 10-100, 10-75, 10-50, 10-35, 10-30, 10-20, 15-100, 15-75, 15-50, 15-35, 15-30, 15-20, 20-100, 20-75, 20-50, 20-35, 20-30, 25-100, 25-75, 25-50, 25-35, 20-30, 35-100, 35-75 or 35-50 amino acids. Each possibility represents a separate embodiment of the invention. In some embodiments, the linker comprises or consists of between 5-35 amino acids. In some embodiments, the linker comprises or consists of between 15-35 amino acids. In some embodiments, the linker comprises or consists of between 5-50 amino acids. In some embodiments, the linker comprises or consists of between 15-50 amino acids. In some embodiments, the linker comprises or consists of between 35 and 50 amino acids. In some embodiments, the linker comprises or consists of between 10 and 20 amino acids. In some embodiments, the linker comprises or consists of between 10 and 30 amino acids. In some embodiments, the linker comprises or consists of between 15 and 20 amino acids. In some embodiments, the linker comprises or consists of between 15 and 30 amino acids. In some embodiments, the linker comprises or consists of between 10 and 40 amino acids. In some embodiments, the linker comprises or consists of between 20 and 40 amino acids.

In some embodiments, the C-terminus of the first moiety is linked to the linker. In some embodiments, the N-terminus of the first moiety is linked to the linker. In some embodiments, the C-terminus of the second moiety is linked to the linker. In some embodiments, the N-terminus of the second moiety is linked to the linker. In some embodiments, the C-terminus of the first moiety is linked to the linker and the N-terminus of the second moiety is linked to the linker. In some embodiments, the N-terminus of the first moiety is linked to the linker and the C-terminus of the second moiety is linked to the linker. In some embodiments, the C-terminus of the first moiety is linked to the N-terminus of the peptide linker. In some embodiments, the N-terminus of the first moiety is linked to the C-terminus of the peptide linker. In some embodiments, the C-terminus of the second moiety is linked to the N-terminus of the peptide linker. In some embodiments, the N-terminus of the second moiety is linked to the C-terminus of the peptide linker. In some embodiments, the C-terminus of the first moiety is linked to the N-terminus of the peptide linker and the C-terminus of the peptide linker is linked to the N-Terminus of the second moiety. In some embodiments, the N-terminus of the first moiety is linked to the C-terminus of the peptide linker and the N-terminus of the peptide linker is linked to the C-terminus of the second linker.

As used herein, the term “linked” refers to any method of attachment known in the art by which two moieties are stably connected. In some embodiments, linked is a covalent linkage. In some embodiments, linked is a peptide linkage. In some embodiments, linked is a reversible linkage. In some embodiments, linked is an irreversible linkage. In some embodiments, linked is an amino linkage. In some embodiments, linked is a thiol linkage. In some embodiments, linked is a serine linkage. In some embodiments, the linkage is linkage to a side chain of an amino acid.

In some embodiments, the linker comprises a cysteine. In some embodiments, the linker comprises at least one cysteine. In some embodiments, the first moiety is linked to the N-terminus of the linker and the cysteine is at the C-terminus of the linker. In some embodiments, the first moiety is linked to the C-terminus of the linker and the cysteine is at the N-terminus of the linker. In some embodiments, the linker comprising the cysteine comprises a histidine tag. In some embodiments, the histidine tag comprises a plurality of histidines. In some embodiments, the histidine tag comprises at least 1, 2, 3, 4, 5, 6, 7, 8, 9, or 10 histidines. Each possibility represents a separate embodiment of the invention. In some embodiments, the histidine tag comprises 6 histidines.

In some embodiments, a PEG molecule is attached to the cysteine. In some embodiments, the second moiety is attached to the cysteine. In some embodiments, the second moiety is attached to the cysteine via thiol linkage. In some embodiments, the second moiety comprises a thiol reactive group or a plurality of thiol reactive groups. In some embodiments, the second moiety comprises at least one thiol reactive group. Examples of thiol reactive groups include, but are not limited to, OPSS, maleimide, vinylsulfone and iodoacetamide functional groups. In some embodiments, the PEG molecule or molecules are attached to the cysteine via thiol linkage. In some embodiments, the PEG molecule or molecules comprise a thiol reactive group or a plurality of thiol reactive groups. In some embodiments, the PEG molecule or molecules comprises one thiol reactive group. In some embodiments, the thiol reactive groups are selected from OPSS, maleimide, vinylsulfone and iodoacetamide functional groups.

In some embodiments, increasing stability is increasing stability in a physiological fluid. In some embodiments, the physiological fluid is a biological fluid. In some embodiments, the bodily fluid is selected from the group consisting of: blood, serum, gastric fluid, intestinal fluid, saliva, bile, tumor fluid, breast milk, urine, interstitial fluid, and stool. In some embodiments, the biological fluid is blood. In some embodiments, blood is whole blood or serum. In some embodiments, blood is serum. In some embodiments, increasing stability in blood comprises reducing clearance from blood. In some embodiments, reducing clearance from blood is reducing clearance of the first moiety from blood. In some embodiments, reducing clearance from blood is reducing clearance of the agent from blood. In some embodiments, reducing clearance from blood comprises reducing renal filtration, reducing lysosomal degradation or both. In some embodiments, reducing clearance from blood comprises reducing renal filtration. In some embodiments, reducing clearance from blood comprises reducing lysosomal degradation. In some embodiments, increasing stability comprises increasing half-life of the first moiety. In some embodiments, increasing stability comprises decreasing degradation. In some embodiments, degradation comprises degradation by proteases. In some embodiments, degradation comprises decreasing degradation by a lysosome. In some embodiments, decreasing clearance comprises decreasing the proportion of the first moiety that is filtered by the renal system or the kidney glomerulus. In some embodiments, the second moiety increases stability of the first moiety, decreases clearance of the first moiety, decreases degradation of the first moiety or any combination thereof. Measuring stability of a protein in a subject is well characterized in the art, and any method may be performed. In some embodiments, measurements of molecule concentration in a fluid are made at various time points in order to calculate stability.

It will be understood by a skilled artisan that when the function of the second moiety is referred to as increasing, decreasing, enhancing, reducing or any comparative measure, the comparison is as compared to the first moiety alone. Comparison to the first moiety linked of the linker is also envisioned. In some embodiments, increase is at least a 5, 10, 15, 20, 25, 30 35, 40, 45, 50, 55, 60, 65, 70, 75, 80, 85, 90, 95, 100, 110, 120,125, 130, 140, 150, 175, 200, 250, 300, 350, 400, 450, 500, 600, 700, 800, 900 or 1000% increase. Each possibility represents a separate embodiment of the invention. In some embodiments, decrease is at least a 5, 10, 15, 20, 25, 30 35, 40, 45, 50, 55, 60, 65, 70, 75, 80, 85, 90, 95, 97, 99 or 100% decrease. Each possibility represents a separate embodiment of the invention.

In some embodiments, the second moiety comprises or consists of a serum or blood molecule. In some embodiments, the serum or blood molecule is a human molecule. In some embodiments, the molecule is a protein. In some embodiments, the protein can be bound by a receptor on a cell. In some embodiments, binding by the receptor enables uptake into the cell. In some embodiments, uptake enables release back into blood. In some embodiments, the protein is serum albumin. In some embodiments, the serum albumin is human serum albumin (HSA). In some embodiments, the second moiety comprises or consists of an HSA polypeptide. In some embodiments, HSA comprises the amino acid sequence DAHKSEVAHREKDLGEENFKALVLIAFAQYLQQCPFEDHVKLVNEVTEFAKTCVADE SAENCDKSLHTLFGDKLCTVATLRETYGEMADCCAKQEPERNECFLQHKDDNPNLPR LVRPEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKAEFA EVSKLVTDLTKVHTECCHGDLLECADDRADLAKYICENQDSISSKLKECCEKPLLEKS HCIAEVENDEMPADLPSLAADFVESKDVCKNYAEAKDVFLGMFLYEYARRHPDYSVV LLLRLAKTYETTLEKCCAAADPHECYAKVFDEFKPLVEEPQNLIKQNCELFEQLGEYK FQNALLVRYTKKVPQVSTPTLVEVSRNLGKVGSKCCKHPEAKRMPCAEDYLSVVLNQ LCVLHEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEGKK LVAASQAALGL (SEQ ID NO: 72). In some embodiments, HSA consists of SEQ ID NO: 72.

In some embodiments, the second moiety comprises or consists of a molecule that binds a serum protein. In some embodiments, the second moiety comprises or consists of a polypeptide that binds a serum protein. In some embodiments, the second moiety comprises or consists of a lipid that binds a serum protein. In some embodiments, the second moiety comprises or consists of a polypeptide that binds a serum albumin. In some embodiments, the second moiety comprises or consists of a polypeptide that binds HSA. In some embodiments, the second moiety comprises or consists of a lipid that binds a serum albumin. In some embodiments, the second moiety comprises or consists of a lipid that binds HSA. In some embodiments, the lipid is a fatty acid or fatty acid derivative. In some embodiments, the second moiety comprises or consists of an HSA binding polypeptide. In some embodiments, the second moiety comprises or consists of an HSA binding lipid. In some embodiments, an HSA binding polypeptide is selected from an antibody, an antigen binding fragment of an antibody, a Fab fragment, a single chain antibody, a single domain antibody, a small molecule and a peptide that specifically binds to HSA. In some embodiments, the HSA binding polypeptide is a Fab fragment. In some embodiments, the HSA binding polypeptide is a single chain antibody. In some embodiments, the HSA binding polypeptide is a single domain antibody. In some embodiments, the HSA binding polypeptide is a peptide that specifically binds to HSA.

In some embodiments, the HSA binding polypeptide comprises a single domain antibody. In some embodiments, the HSA binding polypeptide is a single domain antibody. In some embodiments, the second moiety comprises a single domain antibody comprising or consisting of the sequence: EVQLVESGGGLVQPGNSLRLSCAASGFTFSSFGMSWVRQAPGKGLEWVSSISGSGSDT LYADSVKGRFTISRDNAKTTLYLQMNSLRPEDTAVYYCTIGGSLSRSSQGTLVTVSSAA A (SEQ ID NO: 73). In some embodiments, the single domain antibody binding HSA is Alb8.

In some embodiments, the agent is an agent provided in International Patent Application WO2021181396, herein incorporated by reference in its entirety.

In some embodiments, the agent comprises a first single domain (sdAb) and a second sdAb. In some embodiments, the sdAb is a camelid or shark antibody. In some embodiments, the sdAb is a VHH. In some embodiments, the agent comprises two identical sdAbs. In some embodiments, the two sdAbs comprise the same sequence. In some embodiments, the sequence is an amino acid sequence. In some embodiments, the agent comprises two different sdAbs. In some embodiments, the two sdAbs comprise different sequences. In some embodiments, the two sdAbs comprise the same CDRs. In some embodiments, the two sdAbs comprise different CDRs. In some embodiments, the target epitope of the sdAbs is the CD28 stalk region. In some embodiments, the target epitope of the sdAbs is the CD28 cleavage site. In some embodiments, the target epitope is the site of CD28 protease mediated shedding.

In some embodiments, the agent comprises at least two sdAbs. In some embodiments, the agent comprises a plurality of sdAbs. In some embodiments, the agent comprises at least 2, 3, 4, 5, 6, or 7 sdAbs. Each possibility represents a separate embodiment of the invention. In some embodiments, the agent comprises two sdAbs. In some embodiments, the agent comprises a first sdAb and a second sdAb. In some embodiments, the first sdAb and the second sdAb are the same sdAb. In some embodiments, the first sdAb and the second sdAb comprise the same sequence. In some embodiments, the first sdAb and the second sdAb are different sdAbs. In some embodiments, the first sdAb and the second sdAb comprise different sequences.

In some embodiments, the first sdAb and the second sdAb bind the same mCD28 molecule. In some embodiments, the first sdAb and the second sdAb bind a single mCD28 molecule. In some embodiments, the first sdAb and the second sdAb bind different CD28 molecules. In some embodiments, the first sdAb binds a first CD28 molecule and the second sdAb binds a second CD28 molecule. In some embodiments, the first CD28 molecule and the second CD28 molecule are the same molecule. In some embodiments, the first CD28 molecule and the second CD28 molecule are different molecules.

It will be understood by a skilled artisan that any sdAb that binds to mCD28 on cells and inhibits proteolytic cleavage and sCD28 shedding can be employed as an sdAb of the invention. For the purposes of immune stimulation, such as in the context of cancer for example, the sdAb will also be non-antagonistic, or can be antagonistic so long as once introduced into an agent of the invention the antagonistic effect is lost or reduced to acceptable low levels. For the purposes of immune inhibition, such as in the context of autoimmune disease for example, the sdAb may also be antagonistic, or may become antagonistic once part of an agent of the invention.

In some embodiments, the two CD28 binding sdAbs are linked by a linker. In some embodiments, the first sdAb and the second sdAb are linked by a linker. In some embodiments, the first sdAb is linked to the second sdAb by a linker.

In some embodiments, the agent is a matrix metalloprotease inhibitor (MMPi). In some embodiments, an MMP-2 inhibitor, an MMP-13 inhibitor or both, is an MMP-2 inhibitor. In some embodiments, an MMP-2 inhibitor, an MMP-13 inhibitor or both, is an MMP-13 inhibitor. In some embodiments, an MMP-2 inhibitor, an MMP-13 inhibitor or both, is both an MMP-2 inhibitor and an MMP-13 inhibitor. In some embodiments, both is two inhibitors one that inhibits MMP-2 and one that inhibits MMP-13. In some embodiments, both is a single inhibitor that inhibits both MMP-2 and MMP-13.

In some embodiments, the inhibitor inhibits MMP-2 protease activity. In some embodiments, the inhibitor inhibits MMP-2 sheddase activity. In some embodiments, the inhibitor inhibits MMP-2 binding to CD28. In some embodiments, the inhibitor inhibits MMP-2 binding to mCD28. In some embodiments, the inhibitor is specific to MMP-2. As used herein, the term “specific” refers to the inhibitor substantially inhibiting/binding MMP-2, MMP-13 or both and not substantially inhibiting/binding other proteases. In some embodiments, the inhibitor does not inhibit any proteases other than MMP-2. In some embodiments, the inhibitor directly inhibits MMP-2. In some embodiments, the inhibitor binds MMP-2. In some embodiments, the inhibitor specifically binds MMP-2. In some embodiments, the inhibitor does not bind any proteases other than MMP-2. In some embodiments, the inhibitor binds other proteases other than MMP-2 but does not substantially inhibit those other proteases. In some embodiments, non-inhibition is inhibition at or below 50, 45, 40, 35, 30, 25, 20, 15, 10, 9, 8, 7, 6, 5, 4, 3, 2, or 1%. Each possibility represents a separate embodiment of the invention. In some embodiments, the inhibitor is specific to MMP-2 and at least one of ADAM 10 and ADAM 17. In some embodiments, the inhibitor is specific to MMP-2 and at least one of MMP-13, ADAM 10 and ADAM 17. In some embodiments, the inhibitor is specific to MMP-2 and MMP-13. In some embodiments, the inhibitor is specific to MMP-2 cleavage of mCD28. In some embodiments, the inhibitor is specific to MMP-2 induced cleavage of mCD28. In some embodiments, the inhibitor is specific to MMP-2 mediated cleavage of mCD28. In some embodiments, MMP-2 cleavage of mCD28 is between P144 and L145 of CD28. In some embodiments, P144 and L145 are of SEQ ID NO: 1. In some embodiments, MMP-2 cleavage of mCD28 is between P144 and L145 of SEQ ID NO: 1. In some embodiments, MMP-2 cleavage of mCD28 is between P129 and L130 of SEQ ID NO: 2.

In some embodiments, the inhibitor inhibits MMP-13 protease activity. In some embodiments, the inhibitor inhibits MMP-13 sheddase activity. In some embodiments, the inhibitor inhibits MMP-13 binding to CD28. In some embodiments, the inhibitor inhibits MMP-13 binding to mCD28. In some embodiments, the inhibitor is specific to MMP-13. In some embodiments, the inhibitor does not inhibit any proteases other than MMP-13. In some embodiments, the inhibitor directly inhibits MMP-13. In some embodiments, the inhibitor binds MMP-13. In some embodiments, the inhibitor specifically binds MMP-13. In some embodiments, the inhibitor does not bind any proteases other than MMP-13. In some embodiments, the inhibitor binds other proteases other than MMP-13 but does not substantially inhibit those other proteases. In some embodiments, the inhibitor is specific to MMP-13 and at least one of ADAM 10 and ADAM 17. In some embodiments, the inhibitor is specific to MMP-13 and at least one of MMP-2, ADAM 10 and ADAM 17. In some embodiments, the inhibitor is specific to MMP-13 and MMP-9. In some embodiments, inhibitor does not significantly inhibit ADAM10 or ADAM17. In some embodiments, the inhibitor is specific to MMP-13, MMP-2 and MMP-9. In some embodiments, the inhibitor is specific to MMP-13, MMP-2 and MMP-3. In some embodiments, the inhibitor is specific to MMP-13 cleavage of mCD28. In some embodiments, the inhibitor is specific to MMP-13 induced cleavage of mCD28. In some embodiments, the inhibitor is specific to MMP-13 mediated cleavage of mCD28. In some embodiments, MMP-13 cleavage of mCD28 is between P144 and L145 of CD28. In some embodiments, P144 and L145 are of SEQ ID NO: 1. In some embodiments, MMP-13 cleavage of mCD28 is between P144 and L145 of SEQ ID NO: 1. In some embodiments, MMP-13 cleavage of mCD28 is between P129 and L130 of SEQ ID NO: 2.

In some embodiments, only an MMP-2 inhibitor is administered. In some embodiments, only an MMP-13 inhibitor is administered. In some embodiments, an MMP-2 inhibitor and not an MMP-13 inhibitor is administered. In some embodiments, an MMP-13 inhibitor and not an MMP-2 inhibitor is administered. In some embodiments, neither an ADAM10 nor an ADAM17 inhibitor is administered. In some embodiments, an ADAM10 inhibitor is not administered. In some embodiments, an ADAM17 inhibitor is not administered. In some embodiments, only an MMP-2 inhibitor is contacted. In some embodiments, only an MMP-13 inhibitor is contacted. In some embodiments, an MMP-2 inhibitor and not an MMP-13 inhibitor is contacted. In some embodiments, an MMP-13 inhibitor and not an MMP-2 inhibitor is contacted. In some embodiments, neither an ADAM10 nor an ADAM17 inhibitor is contacted. In some embodiments, an ADAM10 inhibitor is not contacted. In some embodiments, an ADAM17 inhibitor is not contacted.

In some embodiments, inhibition is at least a 10, 20, 25, 30, 40, 50, 60, 70, 75, 80, 85, 90, 95, 97, 99 or 100% inhibition. Each possibility represents a separate embodiment of the invention. In some embodiments, inhibition is complete inhibition. In some embodiments, inhibition is blocking. In some embodiments, the inhibition is partial inhibition.

In some embodiments, the inhibitor is a small molecule inhibitor. In some embodiments, the inhibitor is a small molecule inhibitor of MMP-2. Examples of MMP-2 inhibitors include, but are not limited to: ARP-100, Batimastat, Marimastat, Tanomastat, rebimastat, prinomastat, and Neovastat. In some embodiments, the inhibitor is a monoclonal antibody inhibitor of MMP-2. Examples of antibody-based MMP-2 inhibitors include, but are not limited to, DX-2400, SDS3 and SDS4. In some embodiments, the small molecule inhibitor of MMP-2 is ARP-100.

In some embodiments, the inhibitor is a small molecule inhibitor of MMP-13. Examples of MMP-13 inhibitors include, but are not limited to: CL82198 hydrochloride, 4-N,6-N-bis[(4-fluoro-3-methylphenyl)methyl]pyrimidine-4,6-dicarboxamide, and WAY170523. In some embodiments, the inhibitor is a monoclonal antibody inhibitor of MMP-13. Examples of antibody-based MMP-13 inhibitors include, but are not limited to, C-3 and MM0019-12E10.

In some embodiments, the inhibitor is a small molecule inhibitor of MMP-2 and MMP-13. Examples of inhibitors of both proteins include, but are not limited to: PD166793. In some embodiments, the inhibitor is a small molecule inhibitor of MMP-9 and MMP-13. Examples of inhibitors of both proteins include, but are not limited to: N-Hydroxy-1-(4-methoxyphenyl)sulfonyl-4-(4-biphenylcarbonyl)piperazine-2-carboxamide and 3-[(Hydroxyamino)carbonyl]-4-[(4-methoxyphenyl)sulfonyl]-1-piperazinecarboxylic acid phenylmethyl ester (204139-85-5).

In some embodiments, the inhibitor is a small molecule pan MMP inhibitor. Examples of pan-MMP inhibitors include, but are not limited to actinonin, MMP inhibitor V, N′-hydroxy-N-[1-(methylcarbamoyl)-3-phenyl-propyl]-2-(2-methylpropyl)butanediamide and CP471474. In some embodiments, the inhibitor does not inhibit ADAM10. In some embodiments, the inhibitor does not inhibit ADAM17. In some embodiments, the inhibitor does not inhibit ADAM10 or ADAM17. In some embodiments, the inhibitor is not an ADAM inhibitor. Small molecule inhibitors and antibody inhibitors are well known in the art and may be purchased commercially, for examples from Santa Cruz Biotechnology.

In some embodiments, the agent is an MMPi provided in International Patent Application WO2021111441, herein incorporated by reference in its entirety.

In some embodiments, the agent is produced by a method provided in any one of International Patent Applications WO2019175885, WO2020183473, WO2021181396 and WO2021111441, all of which are incorporated herein by reference in their entirety.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of an agent of the invention to a patient in need thereof. Other suitable routes of administration can include parenteral, subcutaneous, intravenous, intramuscular, or intraperitoneal.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “relapse” refers to a worsening of the cancer after a period of improvement. Relapse need not mean that the cancer has returned to the level before treatment, but only that it has worsened in some way from the time it was in remission. In some embodiments, relapse comprises a return of the cancer after the subject was cancer free. In some embodiments, relapse comprises continued growth of the cancer. In some embodiments, relapse comprises growth of the cancer after a stoppage of growth. In some embodiments, relapse comprises an increase in the rate of growth of the cancer. In some embodiments, relapse comprises return of a symptom of the cancer than had stopped. In some embodiments, relapse comprises worsening of a symptom of the cancer than had been improved. Relapse need only be an increase in the severity of the cancer, or severity of symptoms associated with the cancer.

In some embodiments, the relapse is the relapsed cancer. In some embodiments, the relapse is refractive to the therapy that caused the remission. In some embodiments, the relapse is non-responsive to the therapy that caused the remission.

In some embodiments, the method is performed in vivo. In some embodiments, the method is performed ex vivo. In some embodiments, the method is performed in vitro. In some embodiments, the measuring is performed in vivo. In some embodiments, the measuring is performed ex vivo. In some embodiments, the measuring is performed in vitro. In some embodiments, the sample is an in vitro sample. In some embodiments, the sample is an ex vivo sample.

As used herein, the term “about” when combined with a value refers to plus and minus 10% of the reference value. For example, a length of about 1000 nanometers (nm) refers to a length of 1000 nm+- 100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization - A Laboratory Course Manual” CSHL Press (1996); all of which are incorporated by reference. Other general references are provided throughout this document.

### Materials and Methods

Protease Inhibitors—GI254023X (Sigma, Cat. No. SML0789) was added at 1 μM concentration at the start of each experiment. In cellular week-long assays another dose of the inhibitor was added after 3 days at the final concentration.

ELISA—A commercial ELISA kit (R&D, Cat. No. DY342) was used for quantitation of the amount of soluble human CD28.

Isolation of human immune cells—PBMCs were isolated from fresh blood samples of healthy donors using a standard lymphocytes separation medium (MBP, Cat. No. 850494). CD3 cells were isolated from fresh blood samples of healthy donors using RossetteSEP™ Human T cells Enrichment Kit (STEMCELL, Cat. No. 15061) by a negative selection method. All cells were grown in complete RPMI-1640 media supplemented with 10% HI-FCS and pen/strep mixture.

Dendritic cell differentiation—monocytes were cultured at a density of 1×10⁶/mL in RPMI medium with growth factors that was refreshed at day 3 and at day 6. Immature dendritic cells (iDCs) were induced by addition of 50 ng/mL GM-CSF and 20 ng/mL IL-4 for 6 days. When needed, the iDCs were further differentiated into mature dendritic cells by addition of 100 ng/mL LPS for 48 hrs. The generated cell populations were tested for the indicated phenotypes by FACS analysis of relevant markers and by analysis of secretion of characteristic cytokines.

SEB or CMV activation of PBMCs for the generation of soluble CD28—0.1×10⁶ PBMCs were stimulated with 0.5 ng/mL SEB (Sigma, Cat. No. S4881) for 7 days at 37° C. with/without the indicated concentration of various protease inhibitors in a 96 well plate. For CMV stimulation, 0.5×10⁶ PBMCs were stimulated with 0.5 μg/mL CMV peptivator (Milteny Biotec, Cat. No. 130-093-435) for 7 days at 37° C. with/without the indicated concentration of various protease inhibitors in a 96 well plate. Anti-PD-1 (Keytruda, Merck) was added at 5 μg/mL concentration at the beginning of the assay.

Mixed lymphocyte reaction for the generation of soluble CD28 - 1×10⁵ mature DCs were mixed with 5×10⁵ isolated allogenic CD3 T cells for 6 days. Anti-PD-1 (5 μg/mL, Keytruda, Merck) and 0.1 μM anti-sCD28 VHH-Fc fusion were added at the beginning of the assay.

In-vivo tumor study—C57/b WT mice were provided from Jackson laboratories. Human CD28 Tg Mice were developed by GenOway and licensed for usage in this experiment. Mice used in syngeneic tumor experiments were 6-8 weeks of age at the time of tumor cell implant. 5×10\hat{ }5 MC-38 cells were injected subcutaneous, and tumors were allowed to grow for 5 days before mouse anti-mPD-1 (RMP1-14, BioXcell) or isotype control (rat IgG2a, BioXcell) treatments at doses of 10 mg/kg per mice were administered. Serum samples were taken each week from all human CD28 Tg mice for soluble CD28 serum exposure evaluations.

### Example 1

### sCD28 Levels Before Immunotherapy are Predictive of Response

The immunosuppressive effect of sCD28 has already been established (International Patent Application WO2019/175885, herein incorporated by reference in its entirety). Further, it was found that sCD28 is a potent inhibitor of immunotherapy, and that levels of sCD28 are elevated in a number of cancers. Anti-PD1/anti-PD-L1 and CD80 based immunotherapies were all shown to be negatively impacted by sCD28.

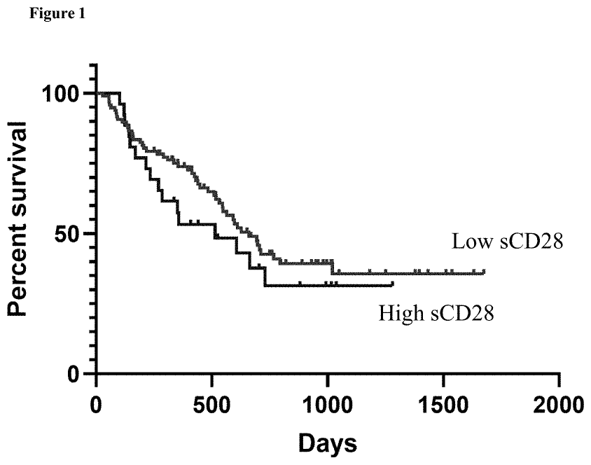

To further understand the impact of sCD28 levels on cancer progression during immunotherapy, more than 100 serum samples from melanoma patients prior to initiation of anti-PD1 therapy (Nivolumab, NCT01176461) were tested for sCD28 levels by ELISA. Survival was monitored throughout therapy and a survival function was plotted comparing subjects with high and low levels of sCD28 before therapy (FIG. 1). It was found that subjects with low levels of sCD28 (below 2 ng/mL) had a longer median survival than those with high levels (659 days vs. 514 days).

### Example 2

### sCD28 Levels During Immunotherapy

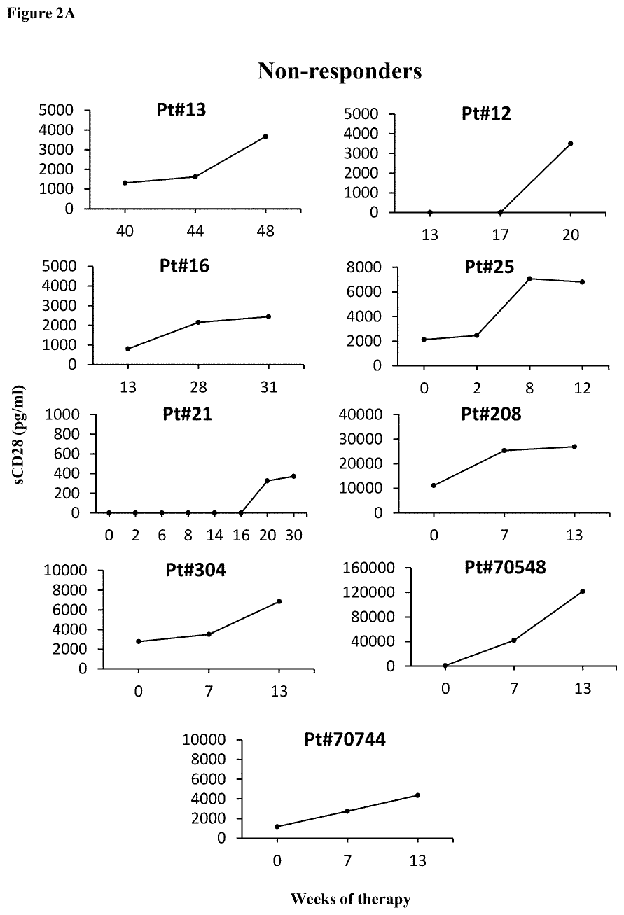

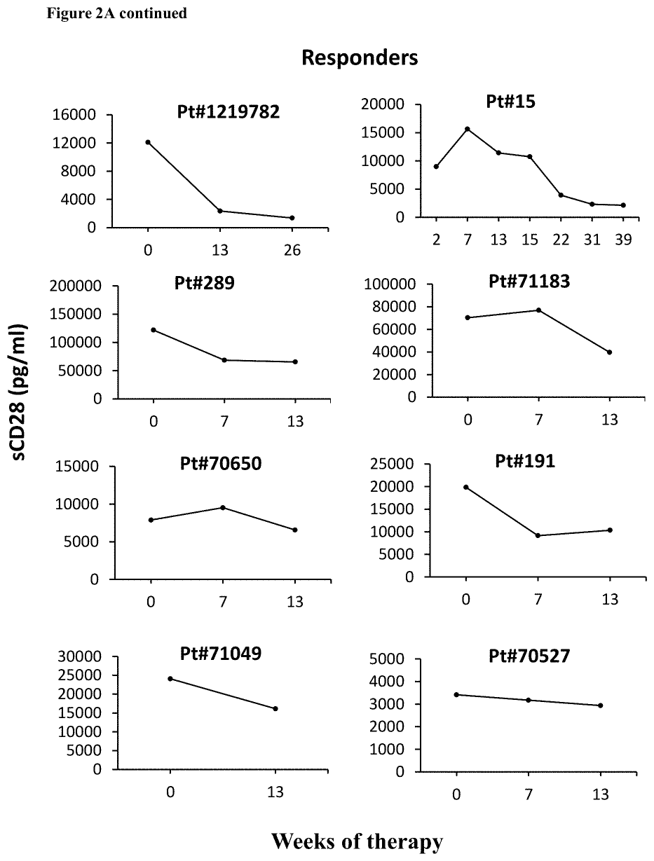

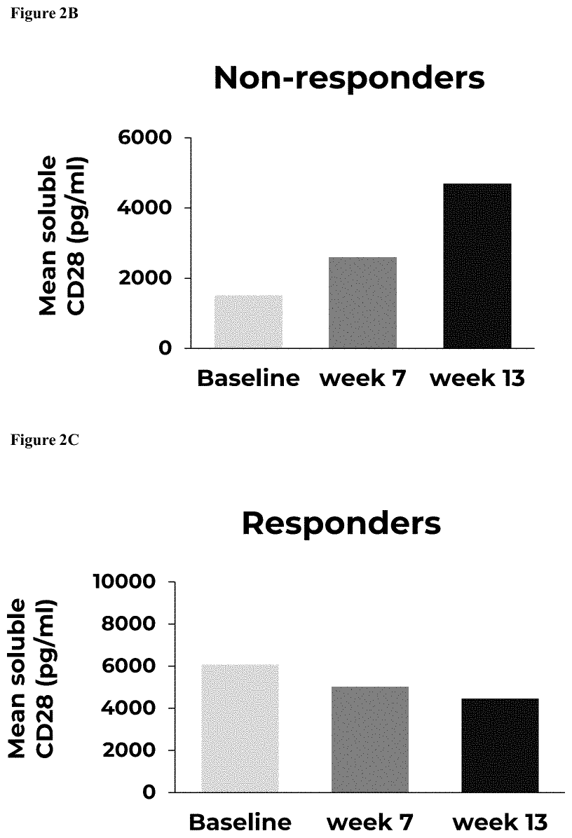

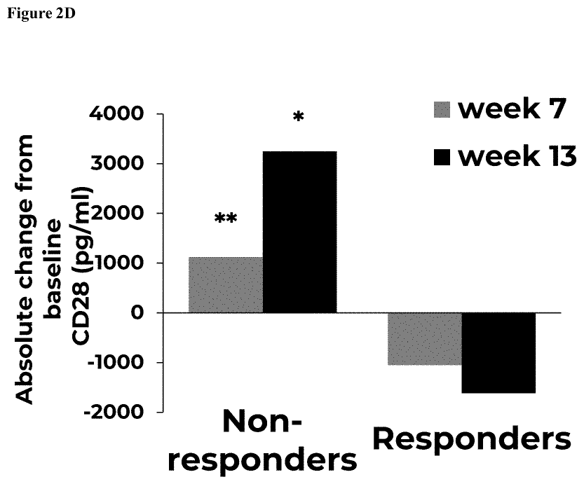

Serum from 166 individual patients (melanoma, renal cell carcinoma, lung squamous cell carcinoma and urothelial carcinoma patient) undergoing immunotherapy (anti-PD-1, for example Nivolumab and Pembrolizumab, anti-PD-L1, for example Atezolizumab and anti-CTLA-4, for example Ipilimumab) were examined for their sCD28 levels. Of these 166, 154 individuals provided multiple samples during the course of therapy. 37 of these subjects were considered to be positive for sCD28 (>2 ng/mL) for at least one time point measured. Of those 37, 19 showed altering kinetics of sCD28 occurrence during therapy (a greater than 50% change in sCD28 levels). Interestingly, the direction of change (positive of negative kinetics) was always consistent: responders had a decrease in sCD28 levels and non-responders showed an increase in sCD28 levels (FIG. 2A).

From these 166 patients, 92 individual patients were undergoing anti-PD-1 therapy with Nivolumab (NCT01176461). All the patients provided samples at three time point (baseline, week 7 and week 13) throughout the course of the immunotherapy. Patients were stratified into responders and non-responders based on cancer remission, as measured by tumor size, at the end of the trial. When the trend in sCD28 levels was examined in the responder and non-responder populations as a whole, the implications of sCD28 as a prognostic marker are readily apparent. Non-responders showed increasing sCD28 levels from baseline (before therapy) to week 7 and through to week 13 (FIG. 2B). In contrast, responders not only did not show increasing levels of sCD28, but in fact the average sCD28 levels decreased from baseline to week 7 and through to week 13 (FIG. 2C). When the absolute change in sCD28 expression is quantified at weeks 7 and 13 the increases seen in the non-responder population were statistically significant (FIG. 2D).

### Example 3

### sCD28 Levels During Relapse

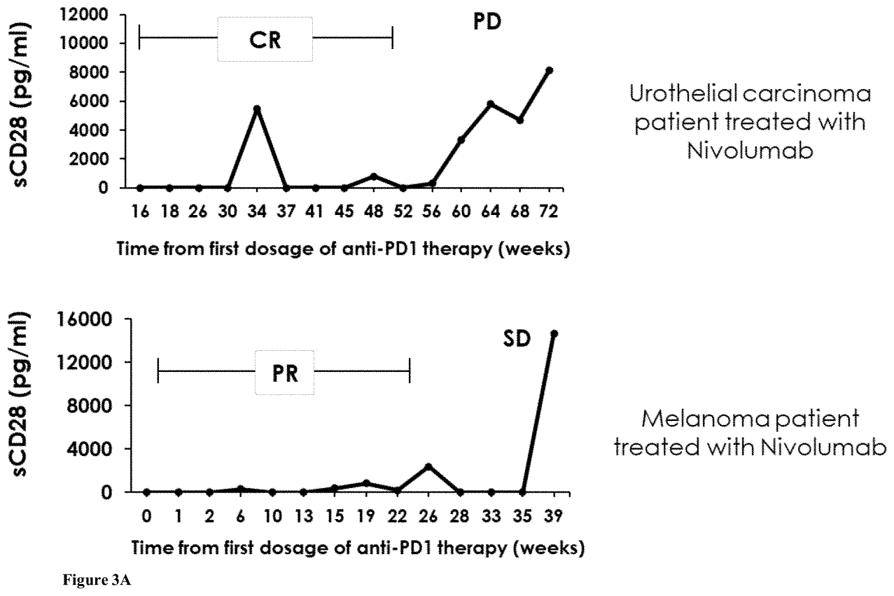

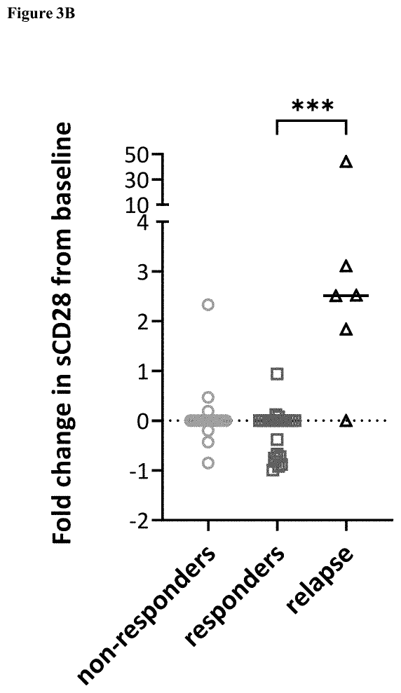

Two of the monitored patients that showed an alteration in kinetics of sCD28 showed an unexpected result. Both patients responded to immunotherapy, with the first (urothelial carcinoma) showing complete response (CR) and the other (melanoma) showing partial response (PR). Both patients were, on average, negative for sCD28 during their response to the therapy, that is during the periods of remission (FIG. 3A). However, both patients showed a marked increase in sCD28 levels just before or at the initiation of cancer relapse. The patient that had a complete response entered a phase of progressive disease (PD) and sCD28 levels began increasing from week 46 and on (FIG. 3A, upper panel). The patient that had a partial response entered a phase of stable disease (SD) and sCD28 levels began increasing at week 35 (FIG. 3A, lower panel).

To further investigate this phenomenon, plasma from 60 individual patients (melanoma, lung cancer, renal cancer, H&N cancer, bladder cancer, mesothelioma and kidney cancer patients) undergoing anti-PD1 immunotherapy (Nivolumab or Pembrolizumab) were examined for their sCD28 levels before and during therapy. The patients' response to therapy was monitored for 12 months after initiation of the therapy and each subject was categorized as either a responder or non-responder to the immunotherapy. FIG. 3B shows the greatest change in sCD28 levels observed during the 6 months for each responders and non-responders. Levels were standardized to the sCD28 levels present before therapy was initiated. Six patients initially responded to the immunotherapy, but eventually relapsed within the first year from therapy initiation. Of these six relapse patients, five already showed increased levels of sCD28 after 6 months of therapy (FIG. 3B, “relapse”). Most of these 5 were still considered “responders” at this point as it was before relapse had been clinically identified. This significant increase in sCD28 levels is thus a highly useful biomarker for predicting imminent cancer relapse. This data taken together demonstrates that increased and/or increasing sCD28 levels can act as a marker for cancer relapse after immunotherapy, and that this increase can be a predictive marker for imminent relapse.

### Example 4

### Reduction of the sCD28 Increase Induced by Immunotherapy

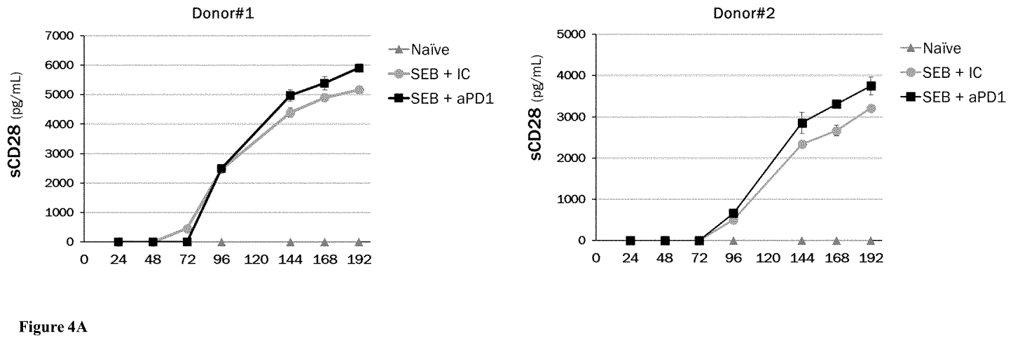

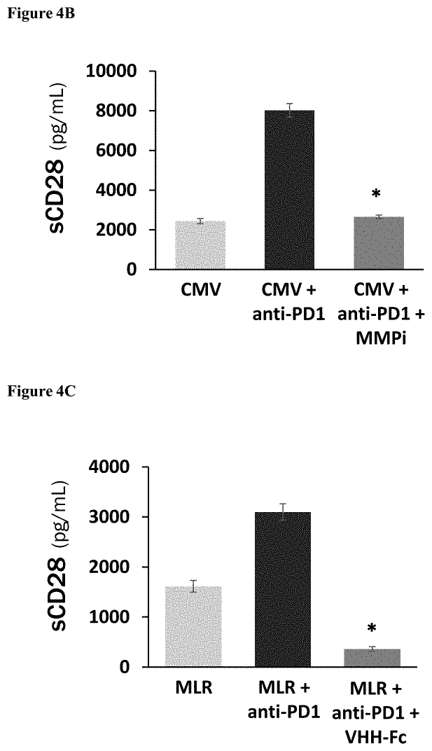

To better understand the relationship between immunotherapy and sCD28, human PBMCs isolated from two healthy donors were stimulated with SEB (super antigen) for a total of 8 days. This stimulation was supplemented with anti-PD-1 (Keytruda, Merck) or isotype control (IC) and the media was sampled every 24 hours for soluble CD28 generation as measured by ELISA. As can be seen in FIG. 4A, the PBMCs from both donors showed the same phenomenon, though SEB alone induced a robust increase in sCD28 in the media, this increase was enhanced by anti-PD-1 treatment. In days 6-8 the increase caused by the immunotherapy was statistically significant.

In order to test whether the sCD28 increase cause by anti-PD-1 was due to increased shedding of the receptor, healthy PBMCs were stimulated with CMV and treated either with IC, anti-PD-1 or anti-PD-1 in the presence of a matrix metalloprotease (MMP) inhibitor (GI254023X). Inhibiting MMP function, which inhibits receptor shedding, abrogated the sCD28 increase caused by immunotherapy (FIG. 4B), thus it is indicated that receptor shedding is increased after anti-PD-1 treatment.

Finally, it was tested if the sCD28 increase could also be inhibited by the addition of a shedding blocking therapeutic agent. An anti-CD28 VHH fused to Fc which is known to bind to the stalk domain of membranal CD28 (mCD28) was used. This VHH (also called 2A1) has been previously shown to inhibit sCD28 shedding. For this experiment a mixed lymphocyte reaction (MLR) was used in order to more closely mimic a physiological setting. Indeed, similar to MMP inhibition, the VHH completely abrogated the sCD28 increase caused by the immunotherapy (FIG. 4C). In fact, it reduced sCD28 levels to even below that of the MLR without addition of anti-PD-1. This demonstrates that agents that inhibit sCD28 levels are effective adjunct therapies to be combined with anti-PD-1/L1 therapies.

### Example 5

### sCD28 Reduces Anti-PD-1 Efficacy

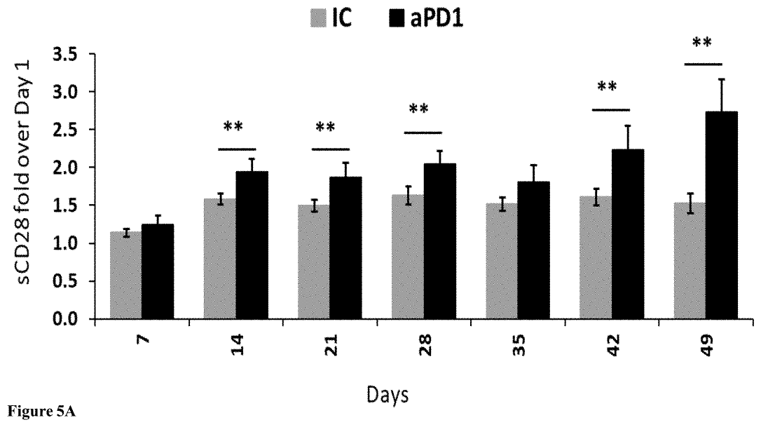

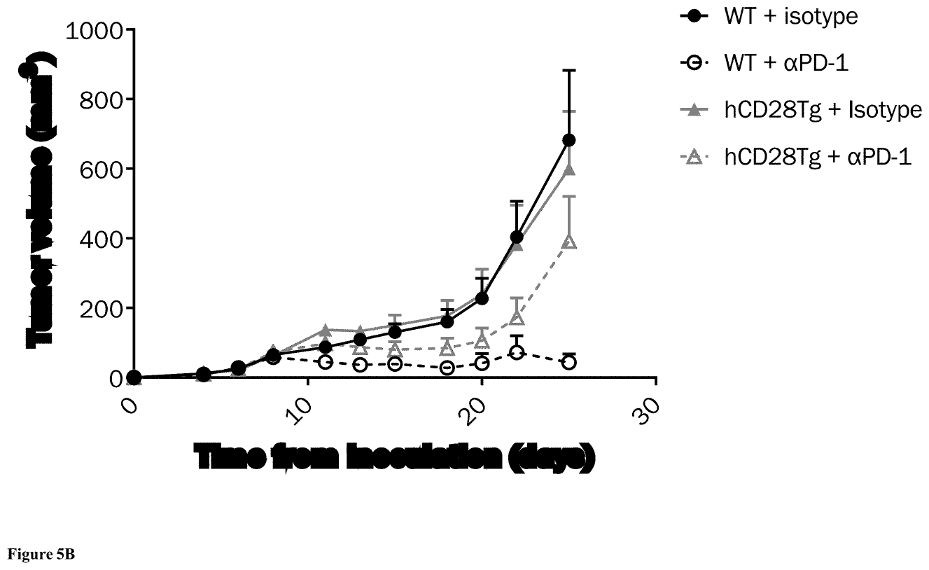

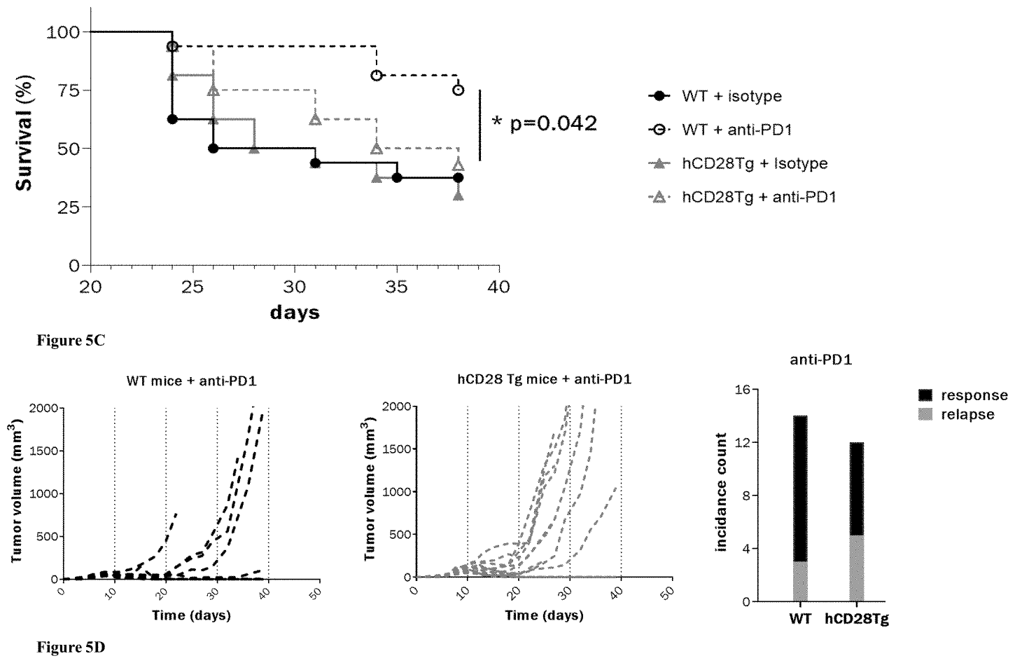

In order to evaluate the immunosuppressive effect of CD28-shedding in an in-vivo model, human CD28 transgenic mice were used, as the CD28 shedding cleavage site is absent in the mouse gene. The efficacy of anti-PD-1 therapy was compared between a syngeneic model (MC38 in C57b strain) in WT mice (devoid of CD28 shedding) and hCD28 transgenic mice (CD28-shedding permissive). Firstly, it was confirmed that the transgenic hCD28 responded to anti-PD-1 treatment in mice as it had in culture: by increased shedding which causes increased serum sCD28 levels (FIG. 5A). Next, the effect of this sCD28 on therapy efficacy was examined in the same mice that had shown the increased shedding. As expected, in the WT mice the anti-PD-1 therapy was highly effective, producing a robust reduction in tumor size (FIG. 5B) and overall survival (FIG. 5C). However, a pronounced reduction in efficacy in the transgenic mice was observed (FIG. 5B-5C). In particular, it was noted that in the transgenic mice anti-PD-1 initially produced a response very similar to that observed in the wild-type mice, however, over time (3-weeks and on) the effect was lost, and tumor size began to increase with a kinetic profile similar to that of the control groups (FIG. 5B). This indicated that there may be mice which are initially responding only to relapse due to increased sCD28 levels. Indeed, when mice were examined individually, relapse cases (mice that experience response—tumor shrinkage over three measurements—and then the tumor started to grow again) were more prevalent in the transgenic mice (FIG. 5D, two left plots). Thus, not only were there fewer transgenic mice that responded to therapy, but a larger percentage of those mice (and a larger number total) relapsed after an initial response (FIG. 5D, right chart).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of treating a subject suffering from cancer or at risk of cancer relapse, the method comprising administering an anti-PD-1/PD-L1 immunotherapy to said subject, measuring soluble CD28 (sCD28) levels in said subject at at least two time points wherein at least a first time point is before said administering and at least a second time point is after said administering, and further administering an agent that reduces sCD28 levels to a subject whose sCD28 levels increased from before administering to after administering by at least a predetermined threshold, thereby treating a subject suffering from cancer.

2. The method of claim 1, wherein said second time point is at least 7 days after said administering.

3. The method of claim 1, wherein an increase in sCD28 levels by more than said predetermined threshold indicates said subject is not responding or unlikely to respond to said anti-PD-1/PD-L1 immunotherapy.

4. The method of claim 3, wherein said administering an agent that reduces sCD28 levels converts a not responding subject to a responding subject.

5. The method of claim 1, wherein said predetermined threshold is 1 ng/ml sCD28, a 10% increase or both.

6. The method of claim 1, further comprising increasing a dose of said PD-1/PD-L1 based immunotherapy administered to said subject.

7. The method of claim 1, wherein said increase is an increase of at least 25%, at least 1 ng/mL sCD28 or both.

8. The method of claim 1, wherein said measuring comprises obtaining a sample from said subject and measuring sCD28 levels in said sample, optionally wherein said sample is a blood sample.

9. The method of claim 1, wherein said cancer is selected from skin cancer, urothelial cancer, lung cancer, colon cancer, head and neck cancer, blader cancer, kidney cancer, mesothelioma and renal cancer.

10. The method of claim 1, wherein said agent that reduces sCD28 levels is an agent that binds sCD28 and degrades said sCD28 or targets said sCD28 for degradation.

11. The method of claim 10, wherein said agent is an antibody or antigen binding fragment thereof and comprises three heavy chain CDRs (CDR-H) and three light chain CDRs (CDR-L), wherein:
CDR-H1 comprises the amino acid sequence set forth in SEQ ID NO: 12 (GYTLTNY), CDR-H2 comprises the amino acid sequence as set forth in SEQ ID NO: 13 (NTYTGK), CDR-H3 comprises the amino acid sequence as set forth in SEQ ID NO: 14 (GDANQQFAY), CDR-L1 comprises the amino acid sequence as set forth in SEQ ID NO: 15 (KASQDINSYLS), CDR-L2 comprises the amino acid sequence as set forth in SEQ ID NO: 16 (RANRLVD), and CDR-L3 comprises the amino acid sequence as set forth in SEQ ID NO: 17 (LQYDEFPPT);
CDR-H1 comprises the amino acid sequence set forth in SEQ ID NO: 18 (GYTFTSY), CDR-H2 comprises the amino acid sequence as set forth in SEQ ID NO: 19 (YPGDGD), CDR-H3 comprises the amino acid sequence as set forth in SEQ ID NO: 20 (NYRYSSFGY), CDR-L1 comprises the amino acid sequence as set forth in SEQ ID NO: 21 (KSSQSLLNSGNQKNYLT), CDR-L2 comprises the amino acid sequence as set forth in SEQ ID NO: 22 (WASTRES), and CDR-L3 comprises the amino acid sequence as set forth in SEQ ID NO: 23 (QSDYSYPLT); or
CDR-H1 comprises the amino acid sequence set forth in SEQ ID NO: 24 (GYTFTDY), CDR-H2 comprises the amino acid sequence as set forth in SEQ ID NO: 25 (NPNYDS), CDR-H3 comprises the amino acid sequence as set forth in SEQ ID NO: 26 (SSPYYDSNHFDY), CDR-L1 comprises the amino acid sequence as set forth in SEQ ID NO: 27 (SARSSINYMH), CDR-L2 comprises the amino acid sequence as set forth in SEQ ID NO: 28 (DTSKLAS), and CDR-L3 comprises the amino acid sequence as set forth in SEQ ID NO: 29 (HQRNSYPFT).

12. The method of claim 1, wherein said agent that reduces sCD28 levels is an agent that binds membranal CD28 (mCD28) and inhibits proteolytic cleavage of said mCD28 and wherein said agent does not degrade mCD28 or decrease mCD28-mediated immune cell activation.

13. The method of claim 12, wherein said agent binds mCD28 in a stalk region and occludes an MMP-2, MMP-13 or both cleavage site, PX₁X₂/X₃ wherein X₃ is a hydrophobic residue, in said stalk region.

14. The method of claim 13, wherein said cleavage site is PSPL and said MMP-2, MMP-13 or both cleaves between said P and said L.

15. The method of claim 12, wherein said agent is an antibody or antigen binding fragment thereof and comprises three heavy chain CDRs (CDR-H) and three light chain CDRs (CDR-L), wherein:
CDR-H1 comprises the amino acid sequence set forth in SEQ ID NO: 30 (GFTFSSYYMS), CDR-H2 comprises the amino acid sequence as set forth in SEQ ID NO:
31 (TISDGGDNTYYAGTVTG), CDR-H3 comprises the amino acid sequence as set forth in SEQ ID NO: 32 (IHWPYYFDS), CDR-L1 comprises the amino acid sequence as set forth in SEQ ID NO: 33 (RASSSVSYMN), CDR-L2 comprises the amino acid sequence as set forth in SEQ ID NO: 34 (ATSDLAS), and CDR-L3 comprises the amino acid sequence as set forth in SEQ ID NO: 35 (QQWSSHPPT); or
a single domain antibody comprising three CDRs wherein:
CDR1 comprises the amino acid sequence set forth in SEQ ID NO: 57 (INAMG),
CDR2 comprises the amino acid sequence as set forth in SEQ ID NO: 58 (AISGGGDTYYADSVKG), CDR3 comprises the amino acid sequence as set forth in SEQ ID NO: 59 (DLYGSDYWD);
CDR1 comprises the amino acid sequence set forth in SEQ ID NO: 36 (INAMA),
CDR2 comprises the amino acid sequence as set forth in SEQ ID NO: 37 (AITSSGSTNYANSVKG), CDR3 comprises the amino acid sequence as set forth in SEQ ID NO: 38 (DEYGSDYWI);
CDR1 comprises the amino acid sequence set forth in SEQ ID NO: 57 (INAMG),
CDR2 comprises the amino acid sequence as set forth in SEQ ID NO: 39 (AITSGGSTNYADSVKG), CDR3 comprises the amino acid sequence as set forth in SEQ ID NO: 40 (DLYGEDYWI);
CDR1 comprises the amino acid sequence set forth in SEQ ID NO: 74 (INSMG),
CDR2 comprises the amino acid sequence as set forth in SEQ ID NO: 75 (AINEKLLIYYADSVKG), CDR3 comprises the amino acid sequence as set forth in SEQ ID NO: 59 (DLYGSDYWD);
CDR1 comprises the amino acid sequence set forth in SEQ ID NO: 57 (INAMG),
CDR2 comprises the amino acid sequence as set forth in SEQ ID NO: 58 (AISGGGDTYYADSVKG), CDR3 comprises the amino acid sequence as set forth in SEQ ID NO: 76 (DMIEQQWWY);
CDR1 comprises the amino acid sequence set forth in SEQ ID NO: 57 (INAMG),
CDR2 comprises the amino acid sequence as set forth in SEQ ID NO: 58 (AISGGGDTYYADSVKG), CDR3 comprises the amino acid sequence as set forth in SEQ ID NO: 77 (DTHRGVYWY);
CDR1 comprises the amino acid sequence set forth in SEQ ID NO: 78 (IKTMA),
CDR2 comprises the amino acid sequence as set forth in SEQ ID NO: 79 (AINYIKEIYYADSVKG), CDR3 comprises the amino acid sequence as set forth in SEQ ID NO: 80 (DVTKEDYWY);
CDR1 comprises the amino acid sequence set forth in SEQ ID NO: 81 (INSMA),
CDR2 comprises the amino acid sequence as set forth in SEQ ID NO: 82 (AISNAREVYYADSVKG), CDR3 comprises the amino acid sequence as set forth in SEQ ID NO: 83 (DVYFQEYWY);
CDR1 comprises the amino acid sequence set forth in SEQ ID NO: 84 (INTMA),
CDR2 comprises the amino acid sequence as set forth in SEQ ID NO: 85 (AINSISRTYYADSVKG), CDR3 comprises the amino acid sequence as set forth in SEQ ID NO: 80 (DVTKEDYWY);
CDR1 comprises the amino acid sequence set forth in SEQ ID NO: 78 (IKTMA),
CDR2 comprises the amino acid sequence as set forth in SEQ ID NO: 86 (AIASDNRKYYADSVKG), CDR3 comprises the amino acid sequence as set forth in SEQ ID NO: 80 (DVTKEDYWY);
CDR1 comprises the amino acid sequence set forth in SEQ ID NO: 87 (IRTMA),
CDR2 comprises the amino acid sequence as set forth in SEQ ID NO: 88 (AISSGREVYYADSVKG), CDR3 comprises the amino acid sequence as set forth in SEQ ID NO: 89 (DMYWQDYWW); or
CDR1 comprises the amino acid sequence set forth in SEQ ID NO: 74 (INSMG),
CDR2 comprises the amino acid sequence as set forth in SEQ ID NO: 90 (AISDRSEKYYADSVKG), CDR3 comprises the amino acid sequence as set forth in SEQ ID NO: 91 (DHHHSDWWT).

16. The method of claim 12, wherein said agent further comprises a moiety that increases stability of said agent.

17. The method of claim 1, wherein said agent that reduces sCD28 levels is a matrix metalloprotease (MMP) inhibitor.

18. The method of claim 17, wherein said MMP is MMP-2, MMP-13 or both.

19. The method of claim 1, wherein said method is a method of treating an imminent cancer relapse and said second time point is while said subject is responding to said immunotherapy.

20. The method of claim 19, wherein imminent cancer relapse is relapse within a year.


SEQUENCE LISTING (109 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 202 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - DNA, 663 bp, Homo sapiens
  source 1..663: mol_type = other DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctga

SEQ ID NO: 4 - protein, 27 aa, Homo sapiens
FWVLVVVGGVLACYSLLVTVAFIIFWV

SEQ ID NO: 5 - protein, 144 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSP

SEQ ID NO: 6 - protein, 126 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSP

SEQ ID NO: 7 - protein, 110 aa, Homo sapiens
MLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKG

SEQ ID NO: 8 - protein, 16 aa, Homo sapiens
GKHLCPSPLFPGPSKP

SEQ ID NO: 9 - protein, 15 aa, Homo sapiens
KGKHLCPSPLFPGPS

SEQ ID NO: 10 - protein, 19 aa, Homo sapiens
HVKGKHLCPSPLFPGPSKP

SEQ ID NO: 11 - protein, 4 aa, Homo sapiens
PSPL

SEQ ID NO: 12 - protein, 7 aa, synthetic construct
GYTLTNY

SEQ ID NO: 13 - protein, 6 aa, synthetic construct
NTYTGK

SEQ ID NO: 14 - protein, 9 aa, synthetic construct
GDANQQFAY

SEQ ID NO: 15 - protein, 11 aa, synthetic construct
KASQDINSYLS

SEQ ID NO: 16 - protein, 7 aa, synthetic construct
RANRLVD

SEQ ID NO: 17 - protein, 9 aa, synthetic construct
LQYDEFPPT

SEQ ID NO: 18 - protein, 7 aa, synthetic construct
GYTFTSY

SEQ ID NO: 19 - protein, 6 aa, synthetic construct
YPGDGD

SEQ ID NO: 20 - protein, 9 aa, synthetic construct
NYRYSSFGY

SEQ ID NO: 21 - protein, 17 aa, synthetic construct
KSSQSLLNSGNQKNYLT

SEQ ID NO: 22 - protein, 7 aa, synthetic construct
WASTRES

SEQ ID NO: 23 - protein, 9 aa, synthetic construct
QSDYSYPLT

SEQ ID NO: 24 - protein, 7 aa, synthetic construct
GYTFTDY

SEQ ID NO: 25 - protein, 6 aa, synthetic construct
NPNYDS

SEQ ID NO: 26 - protein, 12 aa, synthetic construct
SSPYYDSNHFDY

SEQ ID NO: 27 - protein, 10 aa, synthetic construct
SARSSINYMH

SEQ ID NO: 28 - protein, 7 aa, synthetic construct
DTSKLAS

SEQ ID NO: 29 - protein, 9 aa, synthetic construct
HQRNSYPFT

SEQ ID NO: 30 - protein, 10 aa, synthetic construct
GFTFSSYYMS

SEQ ID NO: 31 - protein, 17 aa, synthetic construct
TISDGGDNTYYAGTVTG

SEQ ID NO: 32 - protein, 9 aa, synthetic construct
IHWPYYFDS

SEQ ID NO: 33 - protein, 10 aa, synthetic construct
RASSSVSYMN

SEQ ID NO: 34 - protein, 7 aa, synthetic construct
ATSDLAS

SEQ ID NO: 35 - protein, 9 aa, synthetic construct
QQWSSHPPT

SEQ ID NO: 36 - protein, 5 aa, synthetic construct
INAMA

SEQ ID NO: 37 - protein, 16 aa, synthetic construct
AITSSGSTNYANSVKG

SEQ ID NO: 38 - protein, 9 aa, synthetic construct
DEYGSDYWI

SEQ ID NO: 39 - protein, 16 aa, synthetic construct
AITSGGSTNYADSVKG

SEQ ID NO: 40 - protein, 9 aa, synthetic construct
DLYGEDYWI

SEQ ID NO: 41 - protein, 117 aa, synthetic construct
QIQLVQSGPELKKPGETVKISCKASGYTLTNYGMNWVKQAPGKGLKWMGWINTYTGKPTYVDDFKGRFAFSLETSASTAYLQINNLKNEDTATYFCARGDANQQFAYWGQGTLVTVS

SEQ ID NO: 42 - protein, 442 aa, synthetic construct
QIQLVQSGPELKKPGETVKISCKASGYTLTNYGMNWVKQAPGKGLKWMGWINTYTGKPTYVDDFKGRFAFSLETSASTAYLQINNLKNEDTATYFCARGDANQQFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 43 - protein, 107 aa, synthetic construct
DIKMTQSPSSMYASLGERVTITCKASQDINSYLSWFQQKPGKSPKTLIYRANRLVDGVPSRFSGSGSGQDYSLTISSLEYDDMGIYYCLQYDEFPPTFGAGTKLELK

SEQ ID NO: 44 - protein, 214 aa, synthetic construct
DIKMTQSPSSMYASLGERVTITCKASQDINSYLSWFQQKPGKSPKTLIYRANRLVDGVPSRFSGSGSGQDYSLTISSLEYDDMGIYYCLQYDEFPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 45 - protein, 118 aa, synthetic construct
QVQLQQSGAELARPGASVKLSCKASGYTFTSYWMQWIKKRPGQGLEWIGAIYPGDGDTRYTQKFKGKATLTADKSSTTAYMQLSSLASEDSAVYFCARNYRYSSFGYWGQGTLVTVSA

SEQ ID NO: 46 - protein, 454 aa, synthetic construct
QVQLQQSGAELARPGASVKLSCKASGYTFTSYWMQWIKKRPGQGLEWIGAIYPGDGDTRYTQKFKGKATLTADKSSTTAYMQLSSLASEDSAVYFCARNYRYSSFGYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 47 - protein, 113 aa, synthetic construct
DIVMTQSPSSLTVTAGEKVTLSCKSSQSLLNSGNQKNYLTWYQQKPGQPPQLLIYWASTRESGVPDRFTGSGSGTDFTLTISSVQAEDLAVYYCQSDYSYPLTFGAGTKLELK

SEQ ID NO: 48 - protein, 220 aa, synthetic construct
DIVMTQSPSSLTVTAGEKVTLSCKSSQSLLNSGNQKNYLTWYQQKPGQPPQLLIYWASTRESGVPDRFTGSGSGTDFTLTISSVQAEDLAVYYCQSDYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 49 - protein, 121 aa, synthetic construct
EVQLQQFGAELVKPGASVKISCKASGYTFTDYNMDWVKQSHGKSLEWIGDINPNYDSTAYNQKFMGKATLTVDKSSNTAYMELRSLTSEDTAVYYCARSSPYYDSNHFDYWGQGTSLTVSS

SEQ ID NO: 50 - protein, 445 aa, synthetic construct
EVQLQQFGAELVKPGASVKISCKASGYTFTDYNMDWVKQSHGKSLEWIGDINPNYDSTAYNQKFMGKATLTVDKSSNTAYMELRSLTSEDTAVYYCARSSPYYDSNHFDYWGQGTSLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 51 - protein, 106 aa, synthetic construct
QIVLTQSPAIMSASPGEKVTMTCSARSSINYMHWFQQKPGTSPKRWIYDTSKLASGVPARFSGSGSGTSYSLTISNMEAEDAATYYCHQRNSYPFTFGSGTKLEIK

SEQ ID NO: 52 - protein, 213 aa, synthetic construct
QIVLTQSPAIMSASPGEKVTMTCSARSSINYMHWFQQKPGTSPKRWIYDTSKLASGVPARFSGSGSGTSYSLTISNMEAEDAATYYCHQRNSYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 118 aa, synthetic construct
DVKLVESGGGLVKLGGSLKLSCVASGFTFSSYYMSWVRQTPEKRLEWVATISDGGDNTYYAGTVTGRFTISRDFAKNTLYLQMNSLTSEDTAVYYCARIHWPYYFDSWGQGTTLTVSS

SEQ ID NO: 54 - DNA, 354 bp, synthetic construct
  source 1..354: mol_type = other DNA
gacgtgaagctcgtggagtctgggggaggcttagtgaagcttggagggtccctgaaactctcctgtgtagcctctggattcactttcagtagctattacatgtcttgggttcgccagactccggagaagaggctggagtgggtcgcgaccataagtgatggtggtgataacacctactacgcaggcactgtgacgggccgattcaccatctccagagactttgccaagaacaccctgtacctgcaaatgaacagtctgacctctgaggacacagccgtgtattactgtgcaagaattcattggccttactattttgactcctggggccaaggcaccactctcacagtctcctca

SEQ ID NO: 55 - protein, 106 aa, synthetic construct
QFVLSQSPAILSASPGEMLTMTCRASSSVSYMNWYQQKPGSSPKPWIYATSDLASGVPARFSGSGSGTSYSLTISRVEAEDAATYYCQQWSSHPPTFGGGTKLEIR

SEQ ID NO: 56 - DNA, 318 bp, synthetic construct
  source 1..318: mol_type = other DNA
caatttgttctctcccagtctccagcaatcctgtctgcatctcccggggagatgctcacaatgacttgcagggccagctcaagtgtaagttatatgaactggtatcagcagaagccaggatcttcccccaaaccctggatttatgccacatccgacctggcttctggagtccctgctcgcttcagtggcagtgggtctgggacctcttattctctcacaatcagcagagtggaggctgaagatgctgccacttattactgccagcagtggagtagtcacccacccacgttcggaggggggaccaagctggaaataaga

SEQ ID NO: 57 - protein, 5 aa, synthetic construct
INAMG

SEQ ID NO: 58 - protein, 16 aa, synthetic construct
AISGGGDTYYADSVKG

SEQ ID NO: 59 - protein, 9 aa, synthetic construct
DLYGSDYWD

SEQ ID NO: 60 - protein, 117 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 61 - protein, 117 aa, synthetic construct
EVQLVESGGGLVQAGGSLRLSCAASGSLFSINAMAWYRQAPGKQRELVAAITSSGSTNYANSVKGRFTVSRDNAKNTMYLQMNSLKPEDTAVYYCVVDEYGSDYWIWGQGTQVTVSS

SEQ ID NO: 62 - protein, 117 aa, synthetic construct
QVQLVESGGGLVQAGGSLRLSCAASGSIFSINAMGWYRQAPGKQRERVAAITSGGSTNYADSVKGRFTISRDNAKNTVYLQMNNLEPRDAGVYYCVVDLYGEDYWIWGQGTQVTVSS

SEQ ID NO: 63 - protein, 5 aa, synthetic construct
  VAR_SEQ 5: note = X is G or A
INAMX

SEQ ID NO: 64 - protein, 16 aa, synthetic construct
  VAR_SEQ 3: note = X is S or T
  VAR_SEQ 4: note = X is G or S
  VAR_SEQ 5: note = X is G or S
  VAR_SEQ 7: note = X is D or s
  VAR_SEQ 9: note = X is Y or N
  VAR_SEQ 12: note = X is D or N
AIXXXGXTXYAXSVKG

SEQ ID NO: 65 - protein, 9 aa, synthetic construct
  VAR_SEQ 2: note = X is E or L
  VAR_SEQ 5: note = X is E or S
  VAR_SEQ 9: note = X is D or I
DXYGXDYWX

SEQ ID NO: 66 - protein, 9 aa, synthetic construct
  VAR_SEQ 2: note = X is E or L
  VAR_SEQ 9: note = X is D or I
DXYGSDYWX

SEQ ID NO: 67 - protein, 126 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAHHHHHH

SEQ ID NO: 68 - protein, 126 aa, synthetic construct
EVQLVESGGGLVQAGGSLRLSCAASGSLFSINAMAWYRQAPGKQRELVAAITSSGSTNYANSVKGRFTVSRDNAKNTMYLQMNSLKPEDTAVYYCVVDEYGSDYWIWGQGTQVTVSSAAAHHHHHH

SEQ ID NO: 69 - protein, 126 aa, synthetic construct
QVQLVESGGGLVQAGGSLRLSCAASGSIFSINAMGWYRQAPGKQRERVAAITSGGSTNYADSVKGRFTISRDNAKNTVYLQMNNLEPRDAGVYYCVVDLYGEDYWIWGQGTQVTVSSAAAHHHHHH

SEQ ID NO: 70 - protein, 5 aa, synthetic construct
GGGGS

SEQ ID NO: 71 - protein, 4 aa, synthetic construct
GGGS

SEQ ID NO: 72 - protein, 585 aa, Homo sapiens [sequence omitted]

SEQ ID NO: 73 - protein, 118 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSAAA

SEQ ID NO: 74 - protein, 5 aa, synthetic construct
INSMG

SEQ ID NO: 75 - protein, 16 aa, synthetic construct
AINEKLLIYYADSVKG

SEQ ID NO: 76 - protein, 9 aa, synthetic construct
DMIEQQWWY

SEQ ID NO: 77 - protein, 9 aa, synthetic construct
DTHRGVYWY

SEQ ID NO: 78 - protein, 5 aa, synthetic construct
IKTMA

SEQ ID NO: 79 - protein, 16 aa, synthetic construct
AINYIKEIYYADSVKG

SEQ ID NO: 80 - protein, 9 aa, synthetic construct
DVTKEDYWY

SEQ ID NO: 81 - protein, 5 aa, synthetic construct
INSMA

SEQ ID NO: 82 - protein, 16 aa, synthetic construct
AISNAREVYYADSVKG

SEQ ID NO: 83 - protein, 9 aa, synthetic construct
DVYFQEYWY

SEQ ID NO: 84 - protein, 5 aa, synthetic construct
INTMA

SEQ ID NO: 85 - protein, 16 aa, synthetic construct
AINSISRTYYADSVKG

SEQ ID NO: 86 - protein, 16 aa, synthetic construct
AIASDNRKYYADSVKG

SEQ ID NO: 87 - protein, 5 aa, synthetic construct
IRTMA

SEQ ID NO: 88 - protein, 16 aa, synthetic construct
AISSGREVYYADSVKG

SEQ ID NO: 89 - protein, 9 aa, synthetic construct
DMYWQDYWW

SEQ ID NO: 90 - protein, 16 aa, synthetic construct
AISDRSEKYYADSVKG

SEQ ID NO: 91 - protein, 9 aa, synthetic construct
DHHHSDWWT

SEQ ID NO: 92 - protein, 117 aa, synthetic construct
EVQLVESGGGLVQAGESLRLSCAASGSIASINSMGWYRQAPGSQRELVAAINEKLLIYYADSVKGRFTISRDNAKTTVYLQMNSLRPEDTAVYYCVVDLYGSDYWDWGQGTQVTVSS

SEQ ID NO: 93 - protein, 117 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDMIEQQWWYWGQGTQVTVSS

SEQ ID NO: 94 - protein, 117 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDTHRGVYWYWGQGTQVTVSS

SEQ ID NO: 95 - protein, 117 aa, synthetic construct
EVQLVESGGGLVQAGESLRLSCAASGSIASIKTMAWYRQAPGSQRELVAAINYIKEIYYADSVKGRFTISRDNAKTTVYLQMNSLRPEDTAVYYCVVDVTKEDYWYWGQGTQVTVSS

SEQ ID NO: 96 - protein, 117 aa, synthetic construct
EVQLVESGGGLVQAGESLRLSCAASGSIASINSMAWYRQAPGSQRELVAAISNAREVYYADSVKGRFTISRDNAKTTVYLQMNSLRPEDTAVYYCVVDVYFQEYWYWGQGTQVTVSS

SEQ ID NO: 97 - protein, 117 aa, synthetic construct
EVQLVESGGGLVQAGESLRLSCAASGSIASINTMAWYRQAPGSQRELVAAINSISRTYYADSVKGRFTISRDNAKTTVYLQMNSLRPEDTAVYYCVVDVTKEDYWYWGQGTQVTVSS

SEQ ID NO: 98 - protein, 117 aa, synthetic construct
EVQLVESGGGLVQAGESLRLSCAASGSIASIKTMAWYRQAPGSQRELVTAIASDNRKYYADSVKGRFTISRDNAKTTVYLQMNSLRPEDTAVYYCVVDVTKEDYWYWGQGTQVTVSS

SEQ ID NO: 99 - protein, 117 aa, synthetic construct
EVQLVESGGGLVQPGGSLRLSCAASGSIASIKTMAWYRQAPGKQRELVTAIASDNRKYYADSVKGRFTISRDNSKNTLYLQMNSLRAEDTAVYYCVVDVTKEDYWYWGQGTLVTVSS

SEQ ID NO: 100 - protein, 117 aa, synthetic construct
EVQLVESGGGLVQPGGSLRLSCKASGSIASIKTMAWYRQAPGKGLELVTAIASDNRKYYADSVKGRFTISRDNSKTTVYLQMNSLRAEDTAVYYCVVDVTKEDYWYWGQGTLVTVSS

SEQ ID NO: 101 - protein, 117 aa, synthetic construct
EVQLVESGGGLVQPGGSLRLSCAASGSTASIKTMAWYRQAPGKGLELVTAIASDNRKYYADSVKGRFTISRDNSKTTVYLQMNSLRAEDTAVYYCVVDVTKEDYWYWGQGTLVTVSS

SEQ ID NO: 102 - protein, 117 aa, synthetic construct
EVQLVESGGGLVQPGGSLRLSCKASGSTASIKTMAWYRQAPGKGLELVTAIASDNRKYYADSVKGRFTISRDNSKTTVYLQMNSLRAEDTAVYYCVVDVTKEDYWYWGQGTLVTVSS

SEQ ID NO: 103 - protein, 117 aa, synthetic construct
EVQLVESGGGLVQPGGSLRLSCAASGSIASIKTMAWYRQAPGKGRELVTAIASDNRKYYADSVKGRFTISRDNSKTTVYLQMNSLRAEDTAVYYCVVDVTKEDYWYWGQGTLVTVSS

SEQ ID NO: 104 - protein, 117 aa, synthetic construct
EVQLVESGGGLVQAGESLRLSCAASGSIASIRTMAWYRQAPGSQRELVAAISSGREVYYADSVKGRFTISRDNAKTTVYLQMNSLRPEDTAVYYCVVDMYWQDYWWWGQGTQVTVSS

SEQ ID NO: 105 - protein, 117 aa, synthetic construct
EVQLVESGGGLVQPGESLRLSCAASGSIASIRTMAWYRQAPGSQRELVAAISSGREVYYADSVKGRFTISRDNAKTTVYLQMNSLRAEDTAVYYCVVDMYWQDYWWWGQGTQVTVSS

SEQ ID NO: 106 - protein, 117 aa, synthetic construct
EVQLVESGGGLVQPGGSLRLSCKASGSIASIRTMAWYRQAPGKGLELVAAISSGREVYYADSVKGRFTISRDNSKTTVYLQMNSLRAEDTAVYYCVVDMYWQDYWWWGQGTLVTVSS

SEQ ID NO: 107 - protein, 117 aa, synthetic construct
EVQLVESGGGLVQPGGSLRLSCKASGSTASIRTMAWYRQAPGKGLELVSAISSGREVYYADSVKGRFTISRDNSKTTVYLQMNSLRAEDTAVYYCVVDMYWQDYWWWGQGTLVTVSS

SEQ ID NO: 108 - protein, 117 aa, synthetic construct
EVQLVESGGGLVQPGGSLRLSCAASGSIASIRTMAWYRQAPGKGLELVSAISSGREVYYADSVKGRFTISRDNSKTTVYLQMNSLRAEDTAVYYCVVDMYWQDYWWWGQGTLVTVSS

SEQ ID NO: 109 - protein, 117 aa, synthetic construct
EVQLVESGGGLVQAGESLRLSCAASGSIASINSMGWYRQAPGSQRELVAAISDRSEKYYADSVKGRFTISRDNAKTTVYLQMNSLRPEDTAVYYCVVDHHHSDWWTWGQGTQVTVSS